SEQUENCE LISTING (44 sequences)

SEQ ID NO: 1 - protein, 11 aa, Homo sapiens
  MISC_FEATURE (1)..(1): Xaa is either absent or selected from the group consisting of YLRIVQ, LRIVQ, RIVQ, IVQ, VQ, and Q
  MISC_FEATURE (11)..(11): Xaa is either absent or Phe
XCRSVEGSCGX

SEQ ID NO: 2 - protein, 16 aa, Homo sapiens
YLRIVQCRSVEGSCGF

SEQ ID NO: 3 - protein, 15 aa, Homo sapiens
LRIVQCRSVEGSCGF

SEQ ID NO: 4 - protein, 9 aa, Homo sapiens
CRSVEGSCG

SEQ ID NO: 5 - protein, 10 aa, Homo sapiens
CRSVEGSCGF

SEQ ID NO: 6 - protein, 11 aa, Canine
  MISC_FEATURE (1)..(1): Xaa is absent or selected from the group consisting of YLRVMK, LRVMK, RVMK, VMK, MK, and K
  MISC_FEATURE (11)..(11): Xaa is absent or selected from the group consisting of A and AF
XCRRFVESSCX

SEQ ID NO: 7 - protein, 17 aa, Homo sapiens
YLRVMKCRRFVESSCAF

SEQ ID NO: 8 - protein, 16 aa, Homo sapiens
LRVMKCRRFVESSCAF

SEQ ID NO: 9 - protein, 11 aa, Homo sapiens
CRRFVESSCAF

SEQ ID NO: 10 - protein, 10 aa, Homo sapiens
CRRFVESSCA

SEQ ID NO: 11 - protein, 12 aa, Homo sapiens
  MISC_FEATURE (1)..(1): Xaa is absent or selected from the group consisting of S, HS, GHS, PGHS, APGHS, EAPGHS, SEAPGHS, SSEAPGHS, PSSEAPGHS, DPSSEAPGHS, and IDPSSEAPGHS
  MISC_FEATURE (4)..(4): Xaa is selected from the group consisting of Ser, Ala, Val, Leu, Iso and Gly
  MISC_FEATURE (5)..(5): Xaa is selected from the group consisting of Arg and Lys
  MISC_FEATURE (7)..(7): Xaa is selected from the group consisting of Ser, Ala, Val, Leu, Iso and Gly
  MISC_FEATURE (8)..(8): Xaa is selected from the group consisting of Glu and Asp
  MISC_FEATURE (9)..(10): Xaa is selected from the group consisting of Ser, Ala, Val, Leu, Iso and Gly
  MISC_FEATURE (12)..(12): Xaa is absent or selected from the group consisting of S, SS, SSK, SSKF, SSKFS, SSKFSW, SSKFSWD, SSKFSWDE, SSKFSWDEY, SSKFSWDEYE, SSKFSWDEYEQ, SSKFSWDEYEQY, SSKFSWDEYEQYK, SSKFSWDEYEQYKK, and SSKFSWDEYEQYKKE
XCRXXPXXXXCX

SEQ ID NO: 12 - protein, 2 aa, Homo sapiens
HS

SEQ ID NO: 13 - protein, 3 aa, Homo sapiens
GHS

SEQ ID NO: 14 - protein, 4 aa, Homo sapiens
PGHS

SEQ ID NO: 15 - protein, 5 aa, Homo sapiens
APGHS

SEQ ID NO: 16 - protein, 6 aa, Homo sapiens
EAPGHS

SEQ ID NO: 17 - protein, 7 aa, Homo sapiens
SEAPGHS

SEQ ID NO: 18 - protein, 8 aa, Homo sapiens
SSEAPGHS

SEQ ID NO: 19 - protein, 9 aa, Homo sapiens
PSSEAPGHS

SEQ ID NO: 20 - protein, 10 aa, Homo sapiens
DPSSEAPGHS

SEQ ID NO: 21 - protein, 11 aa, Homo sapiens
IDPSSEAPGHS

SEQ ID NO: 22 - protein, 2 aa, Homo sapiens
SS

SEQ ID NO: 23 - protein, 3 aa, Homo sapiens
SSK

SEQ ID NO: 24 - protein, 4 aa, Homo sapiens
SSKF

SEQ ID NO: 25 - protein, 6 aa, Homo sapiens
CSSKFS

SEQ ID NO: 26 - protein, 6 aa, Homo sapiens
SSKFSW

SEQ ID NO: 27 - protein, 7 aa, Homo sapiens
SSKFSWD

SEQ ID NO: 28 - protein, 8 aa, Homo sapiens
SSKFSWDE

SEQ ID NO: 29 - protein, 9 aa, Homo sapiens
SSKFSWDEY

SEQ ID NO: 30 - protein, 10 aa, Homo sapiens
SSKFSWDEYE

SEQ ID NO: 31 - protein, 11 aa, Homo sapiens
SSKFSWDEYEQ

SEQ ID NO: 32 - protein, 12 aa, Homo sapiens
SSKFSWDEYEQY

SEQ ID NO: 33 - protein, 13 aa, Homo sapiens
SSKFSWDEYEQYK

SEQ ID NO: 34 - protein, 14 aa, Homo sapiens
SSKFSWDEYEQYKK

SEQ ID NO: 35 - protein, 15 aa, Homo sapiens
SSKFSWDEYEQYKKE

SEQ ID NO: 36 - protein, 11 aa, Homo sapiens
SCRSRPVESSC

SEQ ID NO: 37 - protein, 10 aa, Homo sapiens
CRSRPVESSC

SEQ ID NO: 38 - protein, 11 aa, Homo sapiens
CRSRPVESSCS

SEQ ID NO: 39 - protein, 12 aa, Homo sapiens
SCRSRPVESSCS

SEQ ID NO: 40 - protein, 11 aa, Homo sapiens
  MISC_FEATURE (1)..(1): Xaa is absent or selected from the group consisting of YLKLLK, LKLLK, KLLK, LLK, LL and K
  MISC_FEATURE (5)..(5): Xaa is selected from the group consisting of Iso and Val
  MISC_FEATURE (6)..(6): Xaa is selected from the group consisting of His and Tyr
  MISC_FEATURE (7)..(7): Xaa is selected from the group consisting of Asp and Asn
  MISC_FEATURE (8)..(8): Xaa is selected from the group consisting of Asn and Ser
  MISC_FEATURE (11)..(11): Xaa is absent or selected from the group consisting of Gly, and a pharmaceutically acceptable carrier
XCRIXXXXNCX

SEQ ID NO: 41 - protein, 9 aa, Homo sapiens
CRIIHNNNC

SEQ ID NO: 42 - protein, 10 aa, Homo sapiens
CRIIHNNNCG

SEQ ID NO: 43 - protein, 9 aa, Homo sapiens
CRIVYDSNC

SEQ ID NO: 44 - protein, 10 aa, Homo sapiens
CRIVYDSNCG

(19) United States
(12) Patent Application Publication
(10) Pub. No.: US 2022/0160815 A1
(43) Pub. Date: May 26, 2022

(54) PEPTIDES AND USES THEREOF

(71) Applicant: LATERAL IP PTY LTD, Melbourne, Victoria (AU)

(72) Inventor: David KENLEY, Melbourne, Victoria (AU)

(73) Assignee: LATERAL IP PTY LTD, Melbourne, Victoria (AU)

(21) Appl. No.: 17/614,966

(22) PCT Filed: May 29, 2020
(86) PCT No.: PCT/AU2020/050548

§ 371 Date: Nov. 29, 2021

Related U.S. Application Data
(60) Provisional application No. 62/855,270, filed on May 31, 2019.

(30) Foreign Application Priority Data
Jul. 9, 2019 (AU) 2019902436

(51) Int. Cl. A61K 38/08; A61K 38/10; A61P 25/06

(52) U.S. Cl. CPC A61K 38/08; A61P 25/06; A61K 38/10

(57) ABSTRACT

The present disclosure relates to the use of C-terminal fragments of human and non-human growth hormone, synthetic cyclic peptides and C-terminal fragments of human prolactin precursor for the treatment of migraine in a subject in need thereof.

### FIELD OF THE INVENTION

The invention relates generally to peptides useful for treating migraine, and methods of their use, including for alleviating or delaying the onset of migraine and/or symptoms thereof.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Migraine is recognised as a bona fide pain syndrome, alongside the well-accepted neuropathic and nociceptive pain syndromes. While neuropathic pain is caused by damage or injury to the peripheral or central nervous system, and nociceptive pain is caused by damage to somatic or visceral tissue due to trauma or inflammation, migraine is not associated with nerve or tissue injury.

Migraine is a debilitating condition that typically features episodic, recurrent disabling headaches lasting between 4-72 hours, which may be accompanied by other symptoms, such as nausea, vomiting, phonophobia, photophobia, speech disturbances and visual auras. A migraine attack will typically have four phases: 1) the premonitory stage, occurring several hours before a headache, and characterized by symptoms such as fatigue, irritability, difficulty concentrating, mood change, yawning, stiff neck, phonophobia, and/or nausea; 2) the aura phase, with symptoms of sensory or cognitive disturbance; 3) the headache phase comprising throbbing pain, nausea, vomiting and sensory sensitivity; and 4) the postdrome phase, occurring hours to days after resolution of the headache, with symptoms such as weakness, cognitive difficulties, mood changes and gastrointestinal symptoms.

Migraine may be episodic (acute) or chronic. As defined by the The International Classification of Headache Disorders (3^{rd} edition, The International Headache Society, 2018), when migraine occurs on fewer than 15 days per month, it is considered episodic, whereas chronic migraine is typically defined as more than 15 headache days per month over a three month period, of which more than eight are migrainous.

Migraine consistently ranks among the top causes of disability worldwide. Over a billion people worldwide suffer from migraine; and it is cited as the third highest cause of disability in both men and women under the age of 50 in Global Burden of Disease Study 2015. In Australia, 4.9 million people suffer from migraine, and 86% of migraine sufferers are of working age, the total economic cost of migraine in Australia is 35.7 billion, consisting of 14.3 billion of health system costs, 16.3 billion of productivity costs and 5.1 billion of other costs (Deloitte Access Economics, Migraine in Australia Whitepaper, 2018).

A familial susceptibility to migraine has been reported, with further studies confirming genetic predispositions for at least one subtype of migraine. To date, at least three genes associated with migraine susceptibility have been identified: CACNA1A, ATP1A2 and SCNA1.

Migraine occurs more frequently in adult women (18%) than in men (6%), while gender appears to play no role in migraine in children. However, migraine develops most frequently in the second decade, with a peak incidence occurring in adolescence.

Individuals with migraine are also more susceptible to the transient influence of certain triggers, which increase the risk of a migraine attack. These triggers include changes to weather or ambient lighting, certain foods, stress and poor sleep.

Despite the high epidemiological, economic and societal burden, the understanding of migraine pathophysiology remains rudimentary, due in part to a mistaken historical view of migraine as a vascular disorder. Migraine is now recognised as a complex disorder, involving neurogenic inflammation of cranial blood vessels and the abnormal activation and hypersensitisation of several intersecting levels of the nervous system.

There are few effective and specific remedies for treating and preventing migraine. Pharmacological treatment of migraine is typically directed at shortening the duration of attacks or decreasing headache severity or at preventing attacks. Infrequent and less severe migraines may respond to over-the-counter medications such as paracetamol and non-steroidal anti-inflammatory drugs such as aspirin, ibuprofen and naproxen. Current therapies prescribed for moderate to severe/chronic migraines include triptans, Botox, beta blockers, calcium channel blockers, ergotamine and tricyclic antidepressants, opioid and non-opioid analgesics. These medications have significant side effects such as nausea, sedation and sleepiness, and in the case of opioid analgesics, the risk of drug tolerance and drug dependency or addiction. In many instances, migraine drugs have strong contraindications when taken with other medications or controversial potential for increased risks of cardiovascular or prothrombic events. Accordingly, there remains an urgent need for new and alternative options that are effective for the selective treatment of migraine with limited or no side effects. The present invention solves, or partly alleviates, this problem by providing compounds that are effective at alleviating migraine.

### SUMMARY OF THE INVENTION

In an aspect disclosed herein, there is provided a method of treating migraine in a subject, the method comprising administering to a subject a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In another aspect disclosed herein, there is provided a pharmaceutical composition comprising a peptide of formula (I), or a pharmaceutically acceptable salt thereof, for use in the treatment of migraine in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In another aspect disclosed herein, there is provided use of a peptide of formula (I), or a pharmaceutically acceptable salt thereof, in the manufacture of a medicament for the treatment of migraine in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In another aspect disclosed herein, there is provided a method of treating migraine in a subject, the method comprising administering to a subject a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In another aspect disclosed herein, there is provided a pharmaceutical composition comprising a peptide of formula (II), or a pharmaceutically acceptable salt thereof, for use in the treatment of migraine in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In another aspect disclosed herein, there is provided a use of a peptide of formula (II), or a pharmaceutically acceptable salt thereof, in the manufacture of a medicament for the treatment of migraine in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

In another aspect disclosed herein, there is provided a method of treating migraine in a subject, the method comprising administering to a subject a therapeutically effective amount of a peptide of formula (III):

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS,
and
(SEQ ID NO: 21)
IDPSSEAPGHS,

or R¹ is absent; and
R² is selected from the group consisting of

S
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent;
or a pharmaceutically acceptable salt thereof.

In another aspect disclosed herein, there is provided a pharmaceutical composition comprising a peptide of formula (III), or a pharmaceutically acceptable salt thereof, for use in the treatment of migraine in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS,
and
(SEQ ID NO: 21)
IDPSSEAPGHS,

or R¹ is absent; and
R² is selected from the group consisting of

S
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK
(SEQ ID NO: 34)
SSKFSWDEYEQYKK,
and
(SEQ ID NO: 35)
SSKFSWDEYEQYKKE,

or R is absent.

In another aspect disclosed herein, there is provided a use of a peptide of formula (III), or a pharmaceutically acceptable salt thereof, in the manufacture of a medicament for the treatment of migraine in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS,
and
(SEQ ID NO: 21)
IDPSSEAPGHS,

or R¹ is absent; and
R² is selected from the group consisting of

S
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In another aspect disclosed herein, there is provided a method of treating migraine in a subject, the method comprising administering to a subject in need thereof a therapeutically effective amount of a peptide of formula (IV) or a pharmaceutically acceptable salt thereof:

(IV)
(SEQ ID NO: 40)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or
R¹ is absent; and
R² is G (glycine), or R² is absent, or R² is a pharmaceutically acceptable carrier.

In another aspect disclosed herein, there is provided a pharmaceutical composition comprising a peptide of formula (IV) or a pharmaceutically acceptable salt thereof, for use in the treatment of migraine in a subject:

(IV)
(SEQ ID NO: 40)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptable carrier.

In another aspect disclosed herein, there is provided a use of a peptide of formula (IV) or a pharmaceutically acceptable salt thereof, in the manufacture of a medicament for the treatment of migraine in a subject:

(IV)
(SEQ ID NO: 40)
R¹-C-R4-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44).

### BRIEF DESCRIPTION OF THE DRAWINGS

FIGS. 1 to 4 show selected examples of NSR Pain Scores of subjects that completed the study and received treatment with either placebo or LAT8881 (SEQ ID NO:2, 60 mg dose, orally). FIG. 5 shows the mean change from baseline in NRS Pain Score (Bars represent +/−1 standard error).

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

### Peptides of Formula (I)

The present inventor has surprisingly found that peptides of formula (I) (SEQ ID NO:1) can alleviate at least some of the symptoms of migraine. Thus, in an aspect disclosed herein, there is provided a method of treating migraine in a subject, the method comprising administering to a subject a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In a preferred embodiment, the peptide is YLRIVQCRSVEGSCGF (SEQ ID NO:2). SEQ ID NO:2 (also referred to as LAT8881) is the C-terminal fragment of human growth hormone (hGH) spanning amino acid residues 178-192 of hGH (see, e.g., GenBank Accession numbers AAA72260.1, AML27053.1 and ADE06645.1), with an additional tyrosine residue at the N-terminus of the peptide.

In an embodiment disclosed herein, R¹ is absent. In another embodiment, R² is absent. In yet another embodiment, R¹ and R² are absent.

In an embodiment disclosed herein, the peptide of formula (I) is from 9 to 16 amino acid residues in length, preferably 9, 10, 11, 12, 13, 14, 15 or 16 amino acid residues in length. The peptide of formula (I) will typically comprise a disulphide bond between the two cysteine (C) residues, thereby forming a cyclic peptide between the two cysteine residues.

In an embodiment, the peptide of formula (I) is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In a preferred embodiment, the peptide of formula (I) is CRSVEGSCG (SEQ ID NO:4). In another preferred embodiment, the peptide of formula (I) is CRSVEGSCGF (SEQ ID NO:5).

### Peptides of Formula (II)

The present disclosure also extends to non-human variants of the peptides of formula (I) that have therapeutic properties in treating migraine to their human counterparts. Suitable non-human variants of the peptides of formula (I) will be familiar to persons skilled in the art, illustrative examples of which are disclosed in WO 2013/082667, the contents of which is incorporated herein by reference. Thus, in an aspect disclosed herein, there is provided a method of treating migraine in a subject, the method comprising administering to a subject a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent. The peptide of formula (II) is representative of a non-human variant of formula (I), as is found, for example in canine, equine and feline subjects.

In an embodiment, the peptide of formula (II) is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

The peptide of formula (II) is from 9 to 17 amino acid residues in length, preferably 9, 10, 11, 12, 13, 14, 15, 16 or 17 amino acid residues in length. The peptide of formula (II) will typically comprise a disulphide bond between the two cysteine (C) residues, thereby forming a cyclic peptide between the two cysteine residues. In an embodiment, the peptide of formula (II) is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In another embodiment, the peptide is CRRFVESSCAF (SEQ ID NO:9). In another embodiment, the peptide is CRRFVESSCA (SEQ ID NO:10).

### Peptides of Formula (III)

The present disclosure also extends to peptides of formula (III) as having therapeutic properties for the treatment of migraine. Thus, in another aspect disclosed herein, there is provided a method of treating migraine in a subject, the method comprising administering to a subject a therapeutically effective amount of a peptide of formula (III):

(III)
(SEQ ID NO: 1)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS,
and
(SEQ ID NO: 21)
IDPSSEAPGHS,

or R¹ is absent; and
R² is selected from the group consisting of

S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSICFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSICFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In an embodiment, one or both of R¹ and R² further comprises polyethylene glycol (PEG). The PEG may have a molecular weight in the range of 220 to 5500 Da, preferably 220 to 2500 Da, or more preferably 570 to 1100 Da.

In an embodiment, R¹ is absent. In another embodiment, R² is absent. In yet another embodiment, R¹ and R² are absent.

In an embodiment, R is capped with an N-terminal capping group. The term “N-terminal capping group” typically refers to a group that blocks the reactivity of the N-terminal amino group. Suitable N-terminal capping groups will be familiar to persons skilled in the art, illustrative examples of which include acyl groups that form amide groups with the N-terminal amino group, for example, the N-terminal capping group forms a —NHC(O)Ra, where the NH is from the N-terminal amino group and Ra is alkyl, alkenyl, alkynyl, cycloalkyl or aryl. In an embodiment, the N-terminal capping group is —C(O)CH₃ (acyl), forming —NHC(O)CH₃.

In an embodiment, R¹ is a serine residue (S).

In another embodiment, R² is capped with an C-terminal capping group. The term “C-terminal capping group” typically refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR,^{b} where the C(O) is from the C-terminal carboxylic acid group and R^{a} is hydrogen, alkyl, alkenyl, alkynyl, cycloalkyl or aryl and R^{b} is alkyl, alkenyl, alkynyl, cycloalkyl or aryl. In particular embodiments, the C-terminal capping group is —NH₂, forming —C(O)NH₂.

In an embodiment, R² is a serine residue (S).

In another embodiment, R¹ is a serine residue and R² is a serine residue.

The peptides of formula (III) can be from 10 to 50 amino acid residues in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in length), preferably 10 to 40 in length, more preferably 10 to 30 in length, more preferably 10 to 25 in length, or more preferably 10 to 20 in length. It is to be understood that a cyclic peptide, as herein described, is one in which the side chains of two amino acid residues (typically cysteine residues) react together to form a covalent bond or in which the C-terminal carboxylic acid and the N-terminal amine group form an amide bond, thereby cyclizing the peptide.

In an embodiment disclosed herein, the peptide of formula (III) has an amino acid sequence selected from the group consisting of:

(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

### Peptides of Formula (IV)

The present disclosure also extends to peptides of formula (IV) as having therapeutic properties for the treatment of migraine. Thus, in an aspect disclosed herein, there is provided a method of treating migraine in a subject, the method comprising administering to a subject in need thereof a therapeutically effective amount of a peptide of formula (IV) or a pharmaceutically acceptable salt thereof:

(I)
(SEQ ID NO: 40)
R¹-C-R-I-X₁-X₂-X₃-X₄-N-C-R²

wherein
X₁ is an amino acid residue selected from isoleucine (1) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44). In a preferred embodiment, the peptide of formula (IV) is CRIIHNNNC (SEQ ID NO:41). SEQ ID NO:41 (also referred to interchangeably herein as LAT7771) is the C-terminal fragment of human prolactin (PRL) spanning amino acid residues 219-227 of human prolactin precursor (hPRL; see, e.g., NCBI Reference sequence NP_000939.1 and NP_001157030).

The peptides of formulae (I), (II), (III) and (IV) may be made of naturally occurring amino acid residues, proteogenic or non-proteogenic. These amino acids have L-stereochemistry. Naturally occurring amino acids are set out in Table 1, below.

TABLE 1
(1)

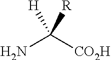

(2)

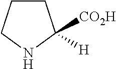

| | Three-letter | One-letter | Structure of side chain |
|---|---|---|---|
| Amino Acid | Abbreviation | symbol | (R) in (1) above |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Ornithine | Orn | O | —(CH₂)₃NH₂ |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “alkyl” refers to a straight chain or branched saturated hydrocarbon group having 1 to 10 carbon atoms. Where appropriate, the alkyl group may have a specified number of carbon atoms, for example, C1-6alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein, the term “alkenyl” refers to a straight-chain or branched hydrocarbon group having one or more double bonds between carbon atoms and having 2 to 10 carbon atoms. Where appropriate, the alkenyl group may have a specified number of carbon atoms. For example, C2-C6 as in “C2-C6alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl and decenyl.

As used herein, the term “alkynyl” refers to a straight-chain or branched hydrocarbon group having one or more triple bonds and having 2 to 10 carbon atoms. Where appropriate, the alkynyl group may have a specified number of carbon atoms. For example, C2-C6 as in “C2-C6alkynyl” includes groups having 2, 3, 4, 5 or 6 carbon atoms in a linear or branched arrangement. Examples of suitable alkynyl groups include, but are not limited to ethynyl, propynyl, butynyl, pentynyl and hexynyl.

As used herein, the term “cycloalkyl” refers to a saturated and unsaturated (but not aromatic) cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentyl, cyclopentenyl, cyclohexyl, cyclohexenyl, cyclohexadienyl, cycloheptyl and cyclooctyl.

As used herein, the term “aryl” is intended to mean any stable, monocyclic, bicyclic or tricyclic carbon ring system of up to 7 atoms in each ring, wherein at least one ring is aromatic. Examples of such aryl groups include, but are not limited to, phenyl, naphthyl, tetrahydronaphthyl, indanyl, fluorenyl, phenanthrenyl, biphenyl and binaphthyl.

In an embodiment, a disulphide bond is formed between the two cysteine residues (C) of formulae (I), (II), (III) and (IV).

The peptides disclosed herein may be made by methods well known to persons skilled in the art, illustrative examples of which include by solution or solid phase synthesis using Fmoc or Boc protected amino acid residues and recombinant techniques as known in the art using standard microbial culture technology, genetically engineered microbes and recombinant DNA technology (Sambrook and Russell, Molecular Cloning: A Laboratory Manual (3d Edition), 2001, CSHL Press).

In an embodiment, the peptides of formulae (I), (II), (III) and (IV) are formed as a pharmaceutically acceptable salt. It is to be understood that non-pharmaceutically acceptable salts are also envisaged, since these may be useful as intermediates in the preparation of pharmaceutically acceptable salts or may be useful during storage or transport. Suitable pharmaceutically acceptable salts will be familiar to persons skilled in the art, illustrative examples of which include salts of pharmaceutically acceptable inorganic acids, such as hydrochloric, sulphuric, phosphoric, nitric, carbonic, boric, sulfamic, and hydrobromic acids, or salts of pharmaceutically acceptable organic acids, such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids. Illustrative examples of suitable base salts include those formed with pharmaceutically acceptable cations, such as sodium, potassium, lithium, calcium, magnesium, ammonium and alkylammonium. Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

Also disclosed herein are prodrugs comprising the peptides of formulae (I), (II), (III) or (IV), or the pharmaceutically acceptable salts thereof. As used herein, a “prodrug” typically refers to a compound that can be metabolized in vivo to provide the active peptide of formulae (I), (II), (III) or (IV), or pharmaceutically acceptable salts thereof. In some embodiments, the prodrug itself also shares the same, or substantially the same, therapeutic activity as the peptide of formulae (I), (II), (III) or (IV), or pharmaceutically acceptable salts thereof, as described elsewhere herein.

In some embodiments, the peptides of formulae (I), (II), (III) and (IV), or pharmaceutically acceptable salts thereof, may further comprise a C-terminal capping group. The term “C-terminal capping group”, as used herein, refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR^{b} where the C(O) is from the C-terminal carboxylic acid group and Ra is hydrogen, alkyl, alkenyl, alkynyl, cycloalkyl or aryl and R is alkyl, alkenyl, alkynyl, cycloalkyl or aryl. In particular embodiments, the C-terminal capping group is —NH₂, forming-C(O)NH₂. In some embodiments, the peptides of formulae (I) or (II), or pharmaceutically acceptable salts thereof, comprise a C-terminal polyethylene glycol (PEG). In an embodiment, the PEG has a molecular weight in the range of 220 to 5500 Da, preferably 220 to 2500 Da, more preferably 570 to 1100 Da.

In some embodiments, the peptides of formulae (I), (II), (III) and (IV), or pharmaceutically acceptable salts thereof, may further comprise an N-terminal capping group. The term “N-terminal capping group”, as used herein, refers to a group that blocks the reactivity of the N-terminal amino group. Suitable N-terminal capping groups are acyl groups that form amide groups with the N-terminal amino group, for example, the N-terminal capping group forms a —NHC(O)Ra where the NH is from the N-terminal amino group and Ra is alkyl, alkenyl, alkynyl, cycloalkyl or aryl. In particular embodiments, the N-terminal capping group is —C(O)CH₃ (acyl), forming —NHC(O)CH₃.

In some embodiments, the peptides of formulae (I), (II), (III) and (IV), or pharmaceutically acceptable salts thereof, may comprise a C-terminal capping group and an N-terminal capping group, as herein described. It is to be understood that the peptides disclosed herein do not include the full length amino acid sequence of human growth hormone or of a non-human isoform thereof.

The peptides of formulae (I), (II), (III) and (IV), or pharmaceutically acceptable salts thereof, as herein described, can be made be any method known to persons skilled in the art. Illustrative examples of suitable methods include solution or solid phase synthesis using Fmoc or Boc protected amino acid residues, recombinant techniques using microbial culture, genetically engineered microbes, plants and recombinant DNA technology (see, e.g., Sambrook and Russell, Molecular Cloning: A Laboratory Manual (3^{rd} Edition), 2001, CSHL Press).

### Methods of Treatment

As described elsewhere herein, the present inventor has surprisingly found, for the first time, that a peptide of formula (I) (SEQ ID NO:1) can alleviate pain and nausea associated with migraine. The peptides of formula (I) can therefore suitably be used to treat, prevent, alleviate or otherwise delay the onset of migraine in a subject, including one or more symptoms of migraine, such as nausea. As disclosed elsewhere herein, the present disclosure also extends to human and non-human variants of formula (II) and formula (III), for treating migraine. Thus, the peptides of formulae (II) and (III), or pharmaceutically acceptable salts thereof, can also suitably be used to treat, prevent, alleviate or otherwise delay the onset of migraine in a subject, including one or more symptoms of migraine, such as nausea. The present disclosure also extends to human and non-human variants of formula (IV) for treating migraine

The terms “treating”, “treatment” and the like, are used interchangeably herein to mean relieving, reducing, alleviating, ameliorating or otherwise inhibiting migraine, including one or more symptoms of migraine, such as nausea. The terms “prevent”, “preventing”, “prophylaxis” and the like are used interchangeably herein to mean reducing the risk of a migraine and one or more symptoms of migraine, such as nausea.

The terms “treating”, “treatment” and the like also include relieving, reducing, alleviating, ameliorating or otherwise inhibiting the effects of migraine for at least a period of time. It is also to be understood that terms “treating”, “treatment” and the like do not imply that the migraine, or a symptom thereof, is permanently relieved, reduced, alleviated, ameliorated or otherwise inhibited and therefore also encompasses the temporary relief, reduction, alleviation, amelioration or otherwise inhibition of migraine, or of one or more symptoms thereof.

As described elsewhere herein, migraine typically features episodic, recurrent disabling headaches lasting between 4-72 hours, which may be accompanied by other symptoms, such as nausea, vomiting, phonophobia, photophobia, speech disturbances and visual auras. A migraine attack will typically have four phases: 1) the premonitory stage, occurring several hours before a headache, and characterized by symptoms such as fatigue, irritability, difficulty concentrating, mood change, yawning, stiff neck, phonophobia, and/or nausea; 2) the aura phase, with symptoms of sensory or cognitive disturbance; 3) the headache phase comprising throbbing pain, nausea, vomiting and sensory sensitivity; and 4) the postdrome phase, occurring hours to days after resolution of the headache, with symptoms such as weakness, cognitive difficulties, mood changes and gastrointestinal symptoms.

Migraine may be episodic (acute) or chronic. As defined by the The International Classification of Headache Disorders (3d edition, The International Headache Society, 2018), when migraine occurs on fewer than 15 days per month, it is considered episodic, whereas chronic migraine is typically defined as more than 15 headache days per month over a three month period, of which more than eight are migrainous.

It is to be understood that contemplated herein is the treatment or prevention of migraine regardless of cause.

In some embodiments, the migraine is accompanied by numbness, weakness and/or loss of reflexes. In some embodiments, the migraine is accompanied by severe and/or disabling pain.

The term “subject”, as used herein, refers to a mammalian subject for whom treatment or prophylaxis of migraine is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In another embodiment, the subject is selected from the group consisting of a canine, a feline and an equine.

It is to be understood that a reference to a subject herein does not imply that the subject has migraine, or a symptom thereof, but also includes a subject that is at risk of developing migraine, or a symptom thereof. In an embodiment, the subject has (i.e., is experiencing) migraine or a symptom thereof. In another embodiment, the subject is not experiencing migraine or a symptom thereof at the time of treatment, but is at risk of developing migraine or a symptom thereof. In an embodiment, the subject suffers from chronic migraine. In another embodiment, the subject suffers from episodic (acute) migraine.

In an embodiment, the methods disclosed herein comprise administering a peptide of formula (I), (II), (III) or (IV), or a pharmaceutically acceptable salt thereof, to a non-human subject. In a preferred embodiment, the non-human subject is selected from the group consisting of a canine, a feline or an equine. In other embodiments, the methods disclosed herein comprise administering a peptide of formula (I), (II), (III) or (IV), or a pharmaceutically acceptable salt thereof, to a human subject. In other embodiments, the peptide of formula (I), (II), (III) or (IV), or a pharmaceutically acceptable salt thereof, is administered to a non-human subject, such as a canine, a feline or an equine.

The peptides of formula (I), (II), (III) and (IV), or pharmaceutically acceptable salts thereof, are to be administered in a therapeutically effective amount. The phrase “therapeutically effective amount” typically means an amount necessary to attain the desired response, or to delay the onset or inhibit progression or halt altogether, the onset or progression of migraine being treated. It would be understood by persons skilled in the art that the therapeutically effective amount of peptide will vary depending upon several factors, illustrative examples of which include the health and physical condition of the subject to be treated, the taxonomic group of subject to be treated, the severity of the migraine to be treated, the formulation of the composition comprising a peptide of formula (I), (II), (III) or (IV), or a pharmaceutically acceptable salt thereof, the route of administration, and combinations of any of the foregoing.

The therapeutically effective amount will typically fall within a relatively broad range that can be determined through routine trials by persons skilled in the art. Illustrative examples of a suitable therapeutically effective amount of the peptides of formulae (I), (II), (III) and (IV), and pharmaceutically acceptable salts thereof, for administration to a human subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. In an embodiment disclosed herein, the therapeutically effective amount of the peptide of formula (I), (II), (III) and/or (IV), and /or pharmaceutically acceptable salts thereof, is from about 0.001 mg per kg of body weight to about 1 g per kg of body weight per dose (e.g., 0.001 mg/kg, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg of body weight, etc). In an embodiment, the therapeutically effective amount of the peptides of formulae (I), (II), (III) and/or (IV), or the pharmaceutically acceptable salts thereof, is from about 0.001 mg to about 50 mg per kg of body weight. In an embodiment, the therapeutically effective amount of the peptide of formula (I), (II), (III) and/or (IV), and pharmaceutically acceptable salts thereof, is from about 0.01 mg to about 100 mg per kg of body weight. In an embodiment, the therapeutically effective amount of the peptide of formula (I), (II), (III) and/or (IV), and pharmaceutically acceptable salts thereof, is from about 0.1 mg to about 10 mg per kg of body weight, preferably from about 0.5 mg to about 5 mg per kg of body weight, more preferably from about 0.5 mg to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

As noted elsewhere herein, the present inventor has surprisingly found that the peptides described herein are capable of alleviating pain and nausea associated with a migraine. Thus, in an embodiment disclosed herein, a peptide of formula (I), or a pharmaceutically acceptable salt thereof, is administered to the subject at a therapeutically effective amount that treats migraine in the subject. Therapeutic activity in treating migraine is also ascribed to the peptides of formulae (II), (III) and (IV). Thus, in an embodiment disclosed herein, a peptide of formula (II), (III) or (IV), or pharmaceutically acceptable salts thereof, is administered to the subject at a therapeutically effective amount that treats migraine in the subject.

In an embodiment disclosed herein, the peptides described herein comprise the amino acid sequence CRSVEGSCG (SEQ ID NO:4) or CRSVEGSCGF (SEQ ID NO:5).

### Routes of Administration

The peptides of formulae (I), (II), (III) and (IV), and pharmaceutically acceptable salts thereof, may be administered to the subject by any suitable route that allows for delivery of the peptides to the subject at a therapeutically effective amount, as herein described. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation). The peptides of formulae (I), (II), (III) and (IV), and pharmaceutically acceptable salts thereof, may also suitably be administered to the subject as a controlled release dosage form to provide a controlled release of the active agent(s) over an extended period of time. The term “controlled release” typically means the release of the active agent(s) to provide a constant, or substantially constant, concentration of the active agent in the subject over a period of time (e.g., about eight hours up to about 12 hours, up to about 14 hours, up to about 16 hours, up to about 18 hours, up to about 20 hours, up to a day, up to a week, up to a month, or more than a month). Controlled release of the active agent(s) can begin within a few minutes after administration or after expiration of a delay period (lag time) after administration, as may be required. Suitable controlled release dosage forms will be known to persons skilled in the art, illustrative examples of which are described in Anal, A. K. (2010; Controlled-Release Dosage Forms. Pharmaceutical Sciences Encyclopedia. 11:1-46).

Without being bound by theory or by a particular mode of application, it may be desirable to elect a route of administration on the basis of the severity of the migraine or one or more symptoms thereof. In an embodiment disclosed herein, the peptides of formulae (I), (II), (III) or (IV), or pharmaceutically acceptable salts thereof, are administered to the subject enterally. In an embodiment disclosed herein, the peptides of formulae (I), (II), (III) or (IV), or pharmaceutically acceptable salts thereof, are administered to the subject orally. In an embodiment disclosed herein, the peptides of formulae (I), (II), (III) or (IV), or pharmaceutically acceptable salts thereof, are administered to the subject parenterally. In another embodiment disclosed herein, the peptides of formulae (I), (II), (III) or (IV), or pharmaceutically acceptable salts thereof, are administered to the subject topically.

As described elsewhere herein, “topical” administration typically means application of the active agents to a surface of the body, such as the skin or mucous membranes, suitably in the form of a cream, lotion, foam, gel, ointment, nasal drop, eye drop, ear drop, transdermal patch, transdermal film (e.g., sublingual film) and the like. Topical administration also encompasses administration via the mucosal membrane of the respiratory tract by inhalation or insufflation. In an embodiment disclosed herein, the topical administration is selected from the group consisting of transdermal and transmucosal administration. In an embodiment, the peptides of formulae (I), (II), (III) and (IV), or pharmaceutically acceptable salts thereof, are administered to the subject transdermally.

In an embodiment, the methods comprise orally administering the peptide of formula (I), or a pharmaceutically acceptable salt thereof, to a human. In another embodiment, the methods comprise orally administering the peptide of formula (I), or pharmaceutically acceptable salts thereof, to a non-human subject. In yet another embodiment, the methods comprise orally administering the peptide of formula (I), or a pharmaceutically acceptable salt thereof, to a non-human subject selected from the group consisting of a feline, a canine and an equine.

In an embodiment, the methods comprise orally administering the peptide of formula (II), or a pharmaceutically acceptable salt thereof, to a human. In another embodiment, the methods comprise orally administering the peptide of formula (II), or a pharmaceutically acceptable salt thereof, to a non-human subject. In yet another embodiment, the methods comprise orally administering the peptide of formula (II), or a pharmaceutically acceptable salt thereof, to a non-human subject selected from the group consisting of a feline, a canine and an equine.

In an embodiment, the methods comprise orally administering the peptide of formula (III), or a pharmaceutically acceptable salt thereof, to a human. In another embodiment, the methods comprise orally administering the peptide of formula (III), or a pharmaceutically acceptable salt thereof, to a non-human subject. In yet another embodiment, the methods comprise orally administering the peptide of formula (III), or a pharmaceutically acceptable salt thereof, to a non-human subject selected from the group consisting of a feline, a canine and an equine.

In an embodiment, the methods comprise orally administering the peptide of formula (IV), or a pharmaceutically acceptable salt thereof, to a human. In another embodiment, the methods comprise orally administering the peptide of formula (IV), or a pharmaceutically acceptable salt thereof, to a non-human subject. In yet another embodiment, the methods comprise orally administering the peptide of formula (IV), or a pharmaceutically acceptable salt thereof, to a non-human subject selected from the group consisting of a feline, a canine and an equine.

In an embodiment, the methods comprise administering the peptide of formula (I), or a pharmaceutically acceptable salt thereof, topically to a human. In another embodiment, the methods comprise administering the peptide of formula (I), or a pharmaceutically acceptable salt thereof, topically to a non-human subject. In yet another embodiment, the methods comprise administering the peptide of formula (I), or a pharmaceutically acceptable salt thereof, topically to a non-human subject selected from the group consisting of a feline, a canine and an equine.

In an embodiment, the methods comprise administering the peptide of formula (II), or a pharmaceutically acceptable salt thereof, topically to a human. In another embodiment, the methods comprise administering the peptide of formula (II), or a pharmaceutically acceptable salt thereof, topically to a non-human subject. In yet another embodiment, the methods comprise administering the peptide of formula (II), or a pharmaceutically acceptable salt thereof, topically to a non-human subject selected from the group consisting of a feline, a canine and an equine.

In an embodiment, the methods comprise administering the peptide of formula (III), or a pharmaceutically acceptable salt thereof, topically to a human. In another embodiment, the methods comprise administering the peptide of formula (III), or a pharmaceutically acceptable salt thereof, topically to a non-human subject. In yet another embodiment, the methods comprise administering the peptide of formula (III), or a pharmaceutically acceptable salt thereof, topically to a non-human subject selected from the group consisting of a feline, a canine and an equine.

In an embodiment, the methods comprise administering the peptide of formula (IV), or a pharmaceutically acceptable salt thereof, topically to a human. In another embodiment, the methods comprise administering the peptide of formula (IV), or a pharmaceutically acceptable salt thereof, topically to a non-human subject. In yet another embodiment, the methods comprise administering the peptide of formula (IV), or a pharmaceutically acceptable salt thereof, topically to a non-human subject selected from the group consisting of a feline, a canine and an equine.

In another embodiment, the methods comprise administering the peptide of SEQ ID NO:2, or a pharmaceutically acceptable salt thereof, orally to a human. In another embodiment, the methods comprise administering the peptide of SEQ ID NO:2, or pharmaceutically acceptable salts thereof, orally to a non-human subject. In yet another embodiment, the methods comprise administering the peptide of SEQ ID NO:2, or pharmaceutically acceptable salts thereof, orally to a non-human subject selected from the group consisting of a feline, a canine and an equine.

In another embodiment, the methods comprise administering the peptide of SEQ ID NO:2, or a pharmaceutically acceptable salt thereof, topically to a human. In another embodiment, the methods comprise administering the peptide of SEQ ID NO:2, or pharmaceutically acceptable salts thereof, topically to a non-human subject. In yet another embodiment, the methods comprise administering the peptide of SEQ ID NO:2, or pharmaceutically acceptable salts thereof, topically to a non-human subject selected from the group consisting of a feline, a canine and an equine.

In another embodiment, the methods comprise administering the peptide of SEQ ID NO:7, or pharmaceutically acceptable salts thereof, orally to a non-human subject. In yet another embodiment, the methods comprise administering the peptide of SEQ ID NO:7, or pharmaceutically acceptable salts thereof, orally to a non-human subject selected from the group consisting of a feline, a canine and an equine.

In another embodiment, the methods comprise administering the peptide of SEQ ID NO:7, or pharmaceutically acceptable salts thereof, topically to a non-human subject. In yet another embodiment, the methods comprise administering the peptide of SEQ ID NO:7, or pharmaceutically acceptable salts thereof, topically to a non-human subject selected from the group consisting of a feline, a canine and an equine.

In another embodiment, the methods comprise administering the peptide of SEQ ID NO:36, or pharmaceutically acceptable salts thereof, orally to a non-human subject. In yet another embodiment, the methods comprise administering the peptide of SEQ ID NO:36, or pharmaceutically acceptable salts thereof, orally to a non-human subject selected from the group consisting of a feline, a canine and an equine.

In another embodiment, the methods comprise administering the peptide of SEQ ID NO:36, or pharmaceutically acceptable salts thereof, topically to a non-human subject. In yet another embodiment, the methods comprise administering the peptide of SEQ ID NO:36, or pharmaceutically acceptable salts thereof, topically to a non-human subject selected from the group consisting of a feline, a canine and an equine.

In another embodiment, the methods comprise administering the peptide of SEQ ID NO:41, or pharmaceutically acceptable salts thereof, orally to a non-human subject. In yet another embodiment, the methods comprise administering the peptide of SEQ ID NO:41, or pharmaceutically acceptable salts thereof, orally to a non-human subject selected from the group consisting of a feline, a canine and an equine.

In another embodiment, the methods comprise administering the peptide of SEQ ID NO:41, or pharmaceutically acceptable salts thereof, topically to a non-human subject. In yet another embodiment, the methods comprise administering the peptide of SEQ ID NO:41, or pharmaceutically acceptable salts thereof, topically to a non-human subject selected from the group consisting of a feline, a canine and an equine.

Illustrative examples of topical administration are described elsewhere herein. In an embodiment, the topical administration is transdermal.

In an embodiment disclosed herein, the peptides of formulae (I) or (II), or pharmaceutically acceptable salts thereof, are administered to the subject as a controlled release dosage form, illustrative examples of which are described elsewhere herein. In an embodiment, the methods comprise administering the peptide of formula (I), or a pharmaceutically acceptable salt thereof, to a human as a controlled release dosage form. In another embodiment, the methods comprise administering the peptide of formula (I), or pharmaceutically acceptable salts thereof, to a non-human subject as a controlled release dosage form. In yet another embodiment, the methods comprise administering the peptide of formula (I), or a pharmaceutically acceptable salt thereof, as a controlled release dosage form to a non-human subject selected from the group consisting of a feline, a canine and an equine.

In another embodiment, the methods comprise administering the peptide of formula (II), or a pharmaceutically acceptable salt thereof, to a human as a controlled release dosage form. In another embodiment, the methods comprise administering the peptide of formula (II), or a pharmaceutically acceptable salt thereof, to a non-human subject as a controlled release dosage form. In yet another embodiment, the methods comprise administering the peptide of formula (II), or a pharmaceutically acceptable salt thereof, as a controlled release dosage form to a non-human subject selected from the group consisting of a feline, a canine and an equine.

In another embodiment, the methods comprise administering the peptide of formula (III), or a pharmaceutically acceptable salt thereof, to a human as a controlled release dosage form. In another embodiment, the methods comprise administering the peptide of formula (III), or a pharmaceutically acceptable salt thereof, to a non-human subject as a controlled release dosage form. In yet another embodiment, the methods comprise administering the peptide of formula (III), or a pharmaceutically acceptable salt thereof, as a controlled release dosage form to a non-human subject selected from the group consisting of a feline, a canine and an equine.

In another embodiment, the methods comprise administering the peptide of formula (IV), or a pharmaceutically acceptable salt thereof, to a human as a controlled release dosage form. In another embodiment, the methods comprise administering the peptide of formula (IV), or a pharmaceutically acceptable salt thereof, to a non-human subject as a controlled release dosage form. In yet another embodiment, the methods comprise administering the peptide of formula (IV), or a pharmaceutically acceptable salt thereof, as a controlled release dosage form to a non-human subject selected from the group consisting of a feline, a canine and an equine.

In another embodiment, the methods comprise administering the peptide of SEQ ID NO:2, or a pharmaceutically acceptable salt thereof, to a human as a controlled release dosage form. In another embodiment, the methods comprise administering the peptide of SEQ ID NO:2, or pharmaceutically acceptable salts thereof, to a non-human subject as a controlled release dosage form. In yet another embodiment, the methods comprise administering the peptide of SEQ ID NO:2, or pharmaceutically acceptable salts thereof, as a controlled release dosage form to a non-human subject selected from the group consisting of a feline, a canine and an equine.

In another embodiment, the methods comprise administering the peptide of SEQ ID NO:7, or pharmaceutically acceptable salts thereof, to a non-human subject as a controlled release dosage form. In yet another embodiment, the methods comprise administering the peptide of SEQ ID NO:7, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:36, or pharmaceutically acceptable salts thereof, to a non-human subject as a controlled release dosage form. In yet another embodiment, the methods comprise administering the peptide of SEQ ID NO:36, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:41, or pharmaceutically acceptable salts thereof, to a non-human subject as a controlled release dosage form. In yet another embodiment, the methods comprise administering the peptide of SEQ ID NO:41, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, it may be beneficial to administer the peptides, as herein disclosed, via more than one route. For example, it may be desirable to administer a first dose parenterally (e.g., via intramuscular, intravenous; subcutaneous, epidural, intra-articular, intraperitoneal, intracisternal or intrathecal routes of administration) to induce a rapid or acute therapeutic effect in a subject, followed by a subsequent (e.g., second, third, fourth, fifth, etc) dose administered enterally (e.g., orally or rectally) and/or topically (e.g., via transdermal or transmucosal routes of administration) to provide continuing availability of the active agent over an extended period subsequent to the acute phase of treatment. Alternatively, it may be desirable to administer a dose enterally (e.g., orally or rectally), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or topically (e.g., via transdermal or transmucosal routes of administration). Alternatively, it may be desirable to administer a dose topically (e.g., via transdermal or transmucosal routes of administration), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or enterally (e.g., orally or rectally).

The route of administration may suitably be selected on the basis of the migraine symptoms, as discussed elsewhere herein. Alternatively, or in addition, the route of administration may suitably be selected having regard to factors such as the subject's general health, age, weight and tolerance (or a lack thereof) for given routes of administration (e.g., where there is a phobia of needles, an alternative route of administration may be selected, such as enteral and/or topical).

It is also to be understood that, where multiple routes of administration are desired, any combination of two or more routes of administration may be used in accordance with the methods disclosed herein. Illustrative examples of suitable combinations include, but are not limited to, (in order of administration), (a) parenteral-enteral; (b) parenteral-topical; (c) parenteral-enteral-topical; (d) parenteral-topical-enteral; (e) enteral-parenteral; (f) enteral-topical; (g) enteral-topical-parenteral; (h) enteral-parenteral-topical; (i) topical-parenteral; (j) topical-enteral; (k) topical-parenteral-enteral; (l) topical-enteral-parenteral; (m) parenteral-enteral-topical-parenteral; (n) parenteral-enteral-topical-enteral; etc.

In an embodiment, the methods comprise (i) parenterally administering to the subject the peptides or compositions, as disclosed herein, and (ii) non-parenterally (i.e, enterally or topically) administering to the subject the peptides or compositions, as disclosed herein, wherein the non-parenteral (enteral or topical) administration is subsequent to the parenteral administration. In an embodiment, the parental administration is selected from the group consisting of intramuscular, a subcutaneous and intravenous. In a further embodiment, the parental administration is subcutaneous. In an embodiment, the non-parental administration is oral.

In an embodiment, the methods disclosed herein comprise (i) parenterally administering to a human subject the peptide of formula (I), or a pharmaceutically acceptable salt thereof, and (ii) orally administering to the human subject the peptide of formula (I), or a pharmaceutically acceptable salt thereof, wherein the oral administration is subsequent to the parenteral administration. In another embodiment, the methods disclosed herein comprise (i) parenterally administering to a human subject the peptide of SEQ ID NO:2, or a pharmaceutically acceptable salt thereof, and (ii) orally administering to the human subject the peptide of SEQ ID NO:2, or a pharmaceutically acceptable salt thereof, wherein the oral administration is subsequent to the parenteral administration. In an embodiment, the parental administration is subcutaneous. In another embodiment, the parental administration is intrathecal.

In an embodiment, the methods disclosed herein comprise (i) parenterally administering to a non-human subject the peptide of formula (II), or a pharmaceutically acceptable salt thereof, and (ii) orally administering to the non-human subject the peptide of formula (II), or a pharmaceutically acceptable salt thereof, wherein the oral administration is subsequent to the parenteral administration. In a further embodiment, the methods disclosed herein comprise (i) parenterally administering to a non-human subject the peptide of SEQ ID NO:7, or a pharmaceutically acceptable salt thereof, and (ii) orally administering to the non-human subject the peptide of SEQ ID NO:7, or a pharmaceutically acceptable salt thereof, wherein the oral administration is subsequent to the parenteral administration. In an embodiment, the non-human subject is selected from the group consisting of a feline, a canine and an equine. In an embodiment, the parental administration is subcutaneous. In another embodiment, the parental administration is intrathecal.

In an embodiment, the methods disclosed herein comprise (i) parenterally administering to a non-human subject the peptide of formula (HI), or a pharmaceutically acceptable salt thereof, and (ii) orally administering to the non-human subject the peptide of formula (III), or a pharmaceutically acceptable salt thereof, wherein the oral administration is subsequent to the parenteral administration. In a further embodiment, the methods disclosed herein comprise (i) parenterally administering to a non-human subject the peptide of SEQ ID NO:36, or a pharmaceutically acceptable salt thereof, and (ii) orally administering to the non-human subject the peptide of SEQ ID NO:36, or a pharmaceutically acceptable salt thereof, wherein the oral administration is subsequent to the parenteral administration. In an embodiment, the non-human subject is selected from the group consisting of a feline, a canine and an equine. In an embodiment, the parental administration is subcutaneous. In another embodiment, the parental administration is intrathecal.

In an embodiment, the methods disclosed herein comprise (i) parenterally administering to a non-human subject the peptide of formula (IV), or a pharmaceutically acceptable salt thereof, and (ii) orally administering to the non-human subject the peptide of formula (IV), or a pharmaceutically acceptable salt thereof, wherein the oral administration is subsequent to the parenteral administration. In a further embodiment, the methods disclosed herein comprise (i) parenterally administering to a non-human subject the peptide of SEQ ID NO:41, or a pharmaceutically acceptable salt thereof, and (ii) orally administering to the non-human subject the peptide of SEQ ID NO:41, or a pharmaceutically acceptable salt thereof, wherein the oral administration is subsequent to the parenteral administration. In an embodiment, the non-human subject is selected from the group consisting of a feline, a canine and an equine. In an embodiment, the parental administration is subcutaneous. In another embodiment, the parental administration is intrathecal.

In a further embodiment, the methods disclosed herein comprise (i) parenterally administering to a human subject the peptide of formula (I), or a pharmaceutically acceptable salt thereof, and (ii) topically administering to the human subject the peptide of formula (I), or a pharmaceutically acceptable salt thereof, wherein the topical administration is subsequent to the parenteral administration. In a further embodiment, the methods disclosed herein comprise (i) parenterally administering to a human subject the peptide of SEQ ID NO:2, or a pharmaceutically acceptable salt thereof, and (ii) topically administering to the human subject the peptide of SEQ ID NO:2, or a pharmaceutically acceptable salt thereof, wherein the topical administration is subsequent to the parenteral administration.

In a further embodiment, the methods disclosed herein comprise (i) parenterally administering to a non-human subject the peptide of formula (II), or a pharmaceutically acceptable salt thereof, and (ii) topically administering to the non-human subject the peptide of formula (II), or a pharmaceutically acceptable salt thereof, wherein the topical administration is subsequent to the parenteral administration. In a further embodiment, the methods disclosed herein comprise (i) parenterally administering to a non-human subject the peptide of SEQ ID NO:7, or a pharmaceutically acceptable salt thereof, and (ii) topically administering to the non-human subject the peptide of SEQ ID NO:7, or a pharmaceutically acceptable salt thereof, wherein the topical administration is subsequent to the parenteral administration.

In a further embodiment, the methods disclosed herein comprise (i) parenterally administering to a non-human subject the peptide of formula (III), or a pharmaceutically acceptable salt thereof, and (ii) topically administering to the non-human subject the peptide of formula (III), or a pharmaceutically acceptable salt thereof, wherein the topical administration is subsequent to the parenteral administration. In a further embodiment, the methods disclosed herein comprise (i) parenterally administering to a non-human subject the peptide of SEQ ID NO:36, or a pharmaceutically acceptable salt thereof, and (ii) topically administering to the non-human subject the peptide of SEQ ID NO:36, or a pharmaceutically acceptable salt thereof, wherein the topical administration is subsequent to the parenteral administration.

In a further embodiment, the methods disclosed herein comprise (i) parenterally administering to a non-human subject the peptide of formula (IV), or a pharmaceutically acceptable salt thereof, and (ii) topically administering to the non-human subject the peptide of formula (IV), or a pharmaceutically acceptable salt thereof, wherein the topical administration is subsequent to the parenteral administration. In a further embodiment, the methods disclosed herein comprise (i) parenterally administering to a non-human subject the peptide of SEQ ID NO:41, or a pharmaceutically acceptable salt thereof, and (ii) topically administering to the non-human subject the peptide of SEQ ID NO:41, or a pharmaceutically acceptable salt thereof, wherein the topical administration is subsequent to the parenteral administration.

In an embodiment, the non-human subject is selected from the group consisting of a feline, a canine and an equine. In an embodiment, the parenteral route of administration is subcutaneous. In another embodiment, the topical route of administration is transdermal. In another embodiment, the parenteral administration is subcutaneous and the topical administration is transdermal.

Alternatively, or in addition, the peptides and compositions as herein described may suitably be administered as a controlled release dosage form. Thus, in an embodiment, the methods comprise (i) parenterally administering to the subject the peptides or compositions, as disclosed herein, and (ii) administering to the subject the peptides or compositions, as disclosed herein, as a controlled release dosage form, wherein the controlled release dosage form is administered subsequent to the parenteral administration. In another embodiment, the methods comprise (i) non-parenterally (enterally or topically) administering to the subject the peptides or compositions, as disclosed herein, and (ii) administering to the subject the peptides or compositions, as disclosed herein, as a controlled release dosage form, wherein the controlled release dosage form is administered to the subject subsequent to the non-parenteral administration. In yet another embodiment, the methods comprise (i) enterally administering to the subject the peptides or compositions, as disclosed herein, and (ii) administering to the subject the peptides or compositions, as disclosed herein, as a controlled release dosage form, wherein the controlled release dosage form is administered to the subject subsequent to the enteral administration. In yet another embodiment, the methods comprise (i) topically administering to the subject the peptides or compositions, as disclosed herein, and (ii) administering to the subject the peptides or compositions, as disclosed herein, as a controlled release dosage form, wherein the controlled release dosage form is administered to the subject subsequent to the topical administration. In a preferred embodiment, the controlled release dosage form is formulated for parenteral administration.

### Pharmaceutical Compositions

The peptides of formulae (I), (II), (III) and (IV), or pharmaceutically acceptable salts thereof, may be formulated for administration to a subject as a neat chemical. However, in certain embodiments, it may be preferable to formulate the peptides of formulae (I), (II), (III) and (IV), or pharmaceutically acceptable salts thereof, as a pharmaceutical composition, including veterinary compositions. Thus, in another aspect disclosed herein, there is provided a pharmaceutical composition comprising a peptide of formula (I), or a pharmaceutically acceptable salt thereof, as described herein, for use in the treatment of migraine in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, the peptide is selected from the group consisting of

(SEQ ID NO: 2)
YLRIVQCRSVEGSCGF,
(SEQ ID NO: 3)
LRIVQCRSVEGSCGF,
(SEQ ID NO: 4)
CRSVEGSCG
and
(SEQ ID NO: 5)
CRSVEGSCGF

In an embodiment, the peptide is YLRIVQCRSVEGSCGF (SEQ ID NO:2). In an embodiment, the peptide is CRSVEGSCG (SEQ ID NO. 4). In an embodiment, the peptide is CRSVEGSCGF (SEQ ID NO:5).

In another aspect disclosed herein, there is provided use of a peptide of formula (I), or a pharmaceutically acceptable salt thereof, as described herein, in the manufacture of a medicament for the treatment of migraine in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5). In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5).

In another aspect disclosed herein, there is provided a pharmaceutical composition comprising a peptide of formula (II), or a pharmaceutically acceptable salt thereof, for use in the treatment of migraine in a subject

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of

(SEQ ID NO: 7)
YLRVMKGRRFVESSCAF,
(SEQ ID NO: 8)
LRVNIKCRRFVESSCAF,
(SEQ ID NO: 9)
CRRFVESSCAF
and
(SEQ ID NO: 10)
CRRFVESSCA.

In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10).

In another aspect disclosed herein, there is provided a use of a peptide of formula (II), or a pharmaceutically acceptable salt thereof, in the manufacture of a medicament for the treatment of migraine in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10).

In another aspect disclosed herein, there is provided a pharmaceutical composition comprising a peptide of formula (III), or a pharmaceutically acceptable salt thereof, for use in the treatment of migraine in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of

S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In another aspect disclosed herein, there is provided use of a peptide of formula (III), or a pharmaceutically acceptable salt thereof, for use in the treatment of migraine in a subject:

(SEQ ID NO: 11)
R¹-C-R-X¹-X²-P-X³-X⁴-X⁵-X⁶-C-R² (III)

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:

S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of

S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In an embodiment disclosed herein, the peptide of formula (III) has an amino acid sequence selected from the group consisting of:

(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

In another aspect disclosed herein, there is provided a pharmaceutical composition comprising a peptide of formula (IV) or a pharmaceutically acceptable salt thereof, for use in the treatment of migraine in a subject:

(SEQ ID NO: 40)
R¹-C-R-I-X₁-X₂-X₃-X₄-N-C-R² (IV)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptable carrier.

In another aspect disclosed herein, there is provided a use of a peptide of formula (IV) or a pharmaceutically acceptable salt thereof, in the manufacture of a medicament for the treatment of migraine in a subject:

(SEQ ID NO: 40)
R¹-C-R-I-X₁-X₂-X₃-X₄-N-C-R² (IV)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44).

As noted elsewhere herein, the peptides of formulae (I), (II), (III) and (IV), or pharmaceutically acceptable salts thereof, may be administered together, either sequentially or in combination (e.g., as an admixture), with one or more other active agents appropriate to the underlying condition to be treated. For example, where the subject has cancer, the compositions disclosed herein may be formulated for administration together, either sequentially or in combination (e.g., as an admixture), with one or more chemotherapeutic agents, illustrative examples of which will be familiar to persons skilled in the art. Combination treatments of this nature can be advantageous, for example, by alleviating pain and nausea accompanying migraine in patients who are undergoing chemotherapy and suffering from a symptom/side effect thereof, such as pain and/or nausea that is often associated with some chemotherapeutic agents.

In an embodiment, the composition further comprises a pharmaceutically acceptable carrier, excipient or diluent, as described elsewhere herein. In an embodiment, the composition is formulated for oral administration.

Illustrative examples of suitable pharmaceutical formulations include those suitable for enteral or parenteral administration, illustrative examples of which are described elsewhere herein, including oral, rectal, buccal, sublingual, vaginal, nasal, topical (e.g., transdermal), intramuscular, subcutaneous, intravenous, epidural, intra-articular and intrathecal.

The peptides of formulae (I), (II), (III) and (IV), or pharmaceutically acceptable salts thereof, may suitably be prepared as pharmaceutical compositions and unit dosage forms to be employed as solids (e.g., tablets or filled capsules) or liquids (e.g., solutions, suspensions, emulsions, elixirs, or capsules filled with the same) for oral use, in the form of ointments, suppositories or enemas for rectal administration, in the form of sterile injectable solutions for parenteral use (e.g., intramuscular, subcutaneous, intravenous, epidural, intra-articular and intrathecal administration); or in the form of ointments, lotions, creams, gels, patches, sublingual strips or films, and the like for parenteral (e.g., topical, buccal, sublingual, vaginal) administration. In an embodiment, the peptides of formulae (I), (II), (III) and (IV), or pharmaceutically acceptable salts thereof, are formulated for topical (e.g., transdermal) delivery. Suitable transdermal delivery systems will be familiar to persons skilled in the art, illustrative examples of which are described by Prausnitz and Langer (2008; Nature Biotechnol. 26(11):1261-1268), the contents of which are incorporated herein by reference. In another embodiment, the peptides of formulae (I), (II), (III) and (IV), or pharmaceutically acceptable salts thereof, are formulated for sublingual or buccal delivery. Suitable sublingual and buccal delivery systems will be familiar to persons skilled in the art, illustrative examples of which include dissolvable strips or films, as described by Bala et al. (2013; Int. J. Pharm. Investig. 3(2):67-76), the contents of which are incorporated herein by reference.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The peptides of formulae (I), (II), (III) and (IV), or pharmaceutically acceptable salts thereof, as described herein, can be formulated for administration in a wide variety of enteral, topical and/or parenteral dosage forms. Suitable dosage forms may comprise, as the active component, either a peptide of formula (I), a peptide of formula (II), a peptide of formula (III), a peptide of formula (IV), pharmaceutically acceptable salts thereof, or combinations of any of the foregoing, as herein described.

In an embodiment, the composition is formulated for oral administration to a human. In another embodiment, the composition is formulated for oral administration to a non-human subject. In yet another embodiment, the composition is formulated for oral administration to a non-human subject selected from the group consisting of a feline, a canine and an equine.

In another embodiment, the composition is formulated for parenteral administration to a human. In another embodiment, the composition is formulated for parenteral administration to a non-human subject. In yet another embodiment, the composition is formulated for parenteral administration to a non-human subject selected from the group consisting of a feline, a canine and an equine. In an embodiment, the parenteral administration is subcutaneous administration.

In another embodiment, the composition is formulated for topical administration to a human. In another embodiment, the composition is formulated for topical administration to a non-human subject. In yet another embodiment, the composition is formulated for topical administration to a non-human subject selected from the group consisting of a feline, a canine and an equine. In an embodiment, the topical administration is transdermal.

In another embodiment, the composition is formulated as a controlled release dosage form to be administered to a human. In another embodiment, the composition is formulated as a controlled release dosage form to be administered to a non-human subject. In yet another embodiment, the composition is formulated as a controlled release dosage form to be administered to a non-human subject selected from the group consisting of a feline, a canine and an equine. Illustrative examples of suitable controlled release dosage forms are described elsewhere herein.

For preparing pharmaceutical compositions of the peptides of formulae (I), (II), (III) and (IV), or pharmaceutically acceptable salts thereof, pharmaceutically acceptable carriers can be either solid or liquid. Illustrative examples of solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier may be a finely divided solid which is in a mixture with the finely divided active component. In tablets, the active component may be mixed with the carrier having the necessary binding capacity in suitable proportions and compacted in the shape and size desired.

In some embodiments, the powders and tablets contain from five or ten to about seventy percent of the active compound. Illustrative examples of suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material, providing a capsule in which the active component, with or without carriers, is surrounded by a carrier. Similarly, cachets and lozenges are also envisaged herein. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The peptides of formulae (I), (II), (III) and (IV), or pharmaceutically acceptable salts thereof, as described herein, may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active compound(s) may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also contemplated herein are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the peptides of formulae (I), (II), (III) and (IV), or pharmaceutically acceptable salts thereof, as described herein, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the peptides used in the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, or in addition, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the peptide will generally have a small particle size for example of the order of 1 to 10 microns or less. Such a particle size may be obtained by means known in the art, for example by micronization.

When desired, formulations adapted to give controlled or sustained release of the active ingredient may be employed, as described elsewhere herein.

In an embodiment, the pharmaceutical preparations, as herein described, are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In an embodiment, the compositions disclosed herein are formulated for oral administration to a human. In yet another embodiment, the compositions disclosed herein are formulated for oral administration to a non-human. In a further embodiment, the compositions disclosed herein are formulated for oral administration to a non-human selected from the group consisting of a feline, a canine and an equine.

In another embodiment, the peptide of formula (I), or a pharmaceutically acceptable salt thereof, as disclosed herein, is formulated for oral administration to a human subject. In another embodiment, the peptide of formula (I), or a pharmaceutically acceptable salt thereof, as disclosed herein, is formulated for oral administration to a non-human subject. In yet another embodiment, the peptide of formula (I), or a pharmaceutically acceptable salt thereof, as disclosed herein, is formulated for oral administration to a non-human subject selected from the group consisting of a feline, a canine and an equine.

In another embodiment, the peptide of formula (I), or a pharmaceutically acceptable salt thereof, as disclosed herein, is formulated for topical administration to a human subject. In yet another embodiment, the peptide of formula (I), or a pharmaceutically acceptable salt thereof, as disclosed herein, is formulated for topical administration to a non-human subject. In another embodiment, the peptide of formula (I), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (I), or a pharmaceutically acceptable salt thereof, as disclosed herein, is formulated for administration to a human subject as a controlled release dosage form. In yet another embodiment, the peptide of formula (I), or a pharmaceutically acceptable salt thereof, as disclosed herein, is formulated for administration to a non-human subject as a controlled release dosage form. In another embodiment, the peptide of formula (I), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (II), or a pharmaceutically acceptable salt thereof, as disclosed herein, are formulated for oral administration to a non-human subject. In yet another embodiment, the peptide of formula (II), or a pharmaceutically acceptable salt thereof, as disclosed herein, is formulated for oral administration to a non-human subject selected from the group consisting of a feline, a canine and an equine.

In another embodiment, the peptide of formula (II), or a pharmaceutically acceptable salt thereof, as disclosed herein, is formulated for topical administration to a non-human subject. In yet another embodiment, the peptide of formula (II), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (II), or a pharmaceutically acceptable salt thereof, as disclosed herein, is formulated for administration to a human subject as a controlled release dosage form. In yet another embodiment, the peptide of formula (It), or a pharmaceutically acceptable salt thereof, as disclosed herein, is formulated for administration to a non-human subject as a controlled release dosage form. In another embodiment, the peptide of formula (II), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (III), or a pharmaceutically acceptable salt thereof, as disclosed herein, are formulated for oral administration to a non-human subject. In yet another embodiment, the peptide of formula (III), or a pharmaceutically acceptable salt thereof, as disclosed herein, is formulated for oral administration to a non-human subject selected from the group consisting of a feline, a canine and an equine.

In another embodiment, the peptide of formula (III), or a pharmaceutically acceptable salt thereof, as disclosed herein, is formulated for topical administration to a non-human subject. In yet another embodiment, the peptide of formula (III), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (III), or a pharmaceutically acceptable salt thereof, as disclosed herein, is formulated for administration to a human subject as a controlled release dosage form. In yet another embodiment, the peptide of formula (III), or a pharmaceutically acceptable salt thereof, as disclosed herein, is formulated for administration to a non-human subject as a controlled release dosage form. In another embodiment, the peptide of formula (III), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (IV), or a pharmaceutically acceptable salt thereof, as disclosed herein, are formulated for oral administration to a non-human subject. In yet another embodiment, the peptide of formula (IV), or a pharmaceutically acceptable salt thereof, as disclosed herein, is formulated for oral administration to a non-human subject selected from the group consisting of a feline, a canine and an equine.

In another embodiment, the peptide of formula (IV), or a pharmaceutically acceptable salt thereof, as disclosed herein, is formulated for topical administration to a non-human subject. In yet another embodiment, the peptide of formula (IV), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:2, or a pharmaceutically acceptable salt thereof, is formulated for oral administration to a human. In another embodiment, the peptide of SEQ ID NO:2, or a pharmaceutically acceptable salt thereof, is formulated for oral administration to a non-human. subject. In yet another embodiment, the peptide of SEQ ID NO:2, or a pharmaceutically acceptable salt thereof, is formulated for oral administration to a non-human subject selected from the group consisting of a feline, a canine and an equine.

In another embodiment, the peptide of SEQ ID NO:2, or a pharmaceutically acceptable salt thereof, as disclosed herein, is formulated for topical administration to a human subject. In yet another embodiment, the peptide of SEQ ID NO:2, or a pharmaceutically acceptable salt thereof, as disclosed herein, is formulated for topical administration to a non-human subject. In another embodiment, the peptide of SEQ ID NO:2, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:2, or a pharmaceutically acceptable salt thereof, as disclosed herein, is formulated for administration to a human subject as a controlled release dosage form. In yet another embodiment, the peptide of SEQ ID NO:2, or a pharmaceutically acceptable salt thereof, as disclosed herein, is formulated for administration to a non-human subject as a controlled release dosage form. In another embodiment, the peptide of SEQ ID NO:2, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:7, or a pharmaceutically acceptable salt thereof, is formulated for oral administration to a non-human. subject. In yet another embodiment, the peptide of SEQ ID NO:7, or a pharmaceutically acceptable salt thereof, is formulated for oral administration to a non-human subject selected from the group consisting of a feline, a canine and an equine.

In another embodiment, the peptide of SEQ ID NO:7, or a pharmaceutically acceptable salt thereof, is formulated for topical administration to a non-human. subject. In yet another embodiment, the peptide of SEQ ID NO:7, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:7, or a pharmaceutically acceptable salt thereof, as disclosed herein, is formulated for administration to a human subject as a controlled release dosage form. In yet another embodiment, the peptide of SEQ ID NO:7, or a pharmaceutically acceptable salt thereof, as disclosed herein, is formulated for administration to a non-human subject as a controlled release dosage form. In another embodiment, the peptide of SEQ ID NO:7, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:36, or a pharmaceutically acceptable salt thereof, is formulated for oral administration to a non-human. subject. In yet another embodiment, the peptide of SEQ ID NO:36, or a pharmaceutically acceptable salt thereof, is formulated for oral administration to a non-human subject selected from the group consisting of a feline, a canine and an equine.

In another embodiment, the peptide of SEQ ID NO:36, or a pharmaceutically acceptable salt thereof, is formulated for topical administration to a non-human. subject. In yet another embodiment, the peptide of SEQ ID NO:36, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:36, or a pharmaceutically acceptable salt thereof, as disclosed herein, is formulated for administration to a human subject as a controlled release dosage form. In yet another embodiment, the peptide of SEQ ID NO:36, or a pharmaceutically acceptable salt thereof, as disclosed herein, is formulated for administration to a non-human subject as a controlled release dosage form. In another embodiment, the peptide of SEQ ID NO:36, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:41, or a pharmaceutically acceptable salt thereof, is formulated for oral administration to a human. In another embodiment, the peptide of SEQ ID NO:41, or a pharmaceutically acceptable salt thereof, is formulated for oral administration to a non-human. subject. In yet another embodiment, the peptide of SEQ ID NO:41, or a pharmaceutically acceptable salt thereof, is formulated for oral administration to a non-human subject selected from the group consisting of a feline, a canine and an equine.

In another embodiment, the peptide of SEQ ID NO:41, or a pharmaceutically acceptable salt thereof, as disclosed herein, is formulated for topical administration to a human subject. In yet another embodiment, the peptide of SEQ ID NO:41, or a pharmaceutically acceptable salt thereof, as disclosed herein, is formulated for topical administration to a non-human subject. In another embodiment, the peptide of SEQ ID NO:41, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:41, or a pharmaceutically acceptable salt thereof, as disclosed herein, is formulated for administration to a human subject as a controlled release dosage form. In yet another embodiment, the peptide of SEQ ID NO:41, or a pharmaceutically acceptable salt thereof, as disclosed herein, is formulated for administration to a non-human subject as a controlled release dosage form. In another embodiment, the peptide of SEQ ID NO:41, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, the compositions disclosed herein can be suitably formulated for administration via said multiple routes. For example, it may be desirable to administer a first dose parenterally (e.g., intramuscular, intravenously; subcutaneously, etc) to induce a rapid or otherwise acute therapeutic effect in a subject, followed by a subsequent (e.g., second, third, fourth, fifth, etc) dose administered non-parenterally (e.g., enterally and/or topically) to provide continuing availability of the active agent over an extended period subsequent to the acute phase of treatment. Thus, in an embodiment, the peptides and compositions, as disclosed herein, are formulated for parenteral administration to the subject as a first dose (i.e., as a parenteral dosage form) and formulated for non-parenteral administration to the subject after the first dose (e.g., as an enteral and/or topical dosage form). In an embodiment, the parental administration is selected from the group consisting of intramuscular, subcutaneous and intravenous. In a further embodiment, the parental administration is subcutaneous.

In another embodiment, the enteral administration is oral administration. Thus, in an embodiment, the peptides and compositions, as disclosed herein, are formulated for parenteral administration to the subject as a first dose and formulated for oral administration to the subject after the first dose (i.e., as an oral dosage form).

In another embodiment, the enteral administration is topical administration. Thus, in an embodiment, the peptides and compositions, as disclosed herein, are formulated for parenteral administration to the subject as a first dose and formulated for topical administration to the subject after the first dose (i.e., as an oral dosage form). In an embodiment, the topical administration is transdermal administration.

In another embodiment, it may be desirable to administer a first dose parenterally (e.g., intramuscular, intravenously; subcutaneously, etc.) to induce a rapid or otherwise acute therapeutic effect in a subject, followed by a subsequent (e.g., second, third, fourth, fifth, etc.) administration of a controlled release dosage form, as described elsewhere herein, to provide a controlled release of the active agent over an extended period subsequent to the acute phase of treatment. Thus, in another embodiment, the peptides and compositions, as disclosed herein, are formulated for parenteral administration to the subject as a first dose and formulated as a controlled release dosage form to be administered to the subject after the first dose. In an embodiment, the controlled release dosage form is formulated for parental administration.

It may also be desirable to administer a first dose enterally (e.g., orally or rectally), followed by a subsequent (e.g., second, third, fourth, fifth, etc) dose administered topically (e.g., transdermally). Thus, in an embodiment, the peptides and compositions, as disclosed herein, are formulated for enteral administration to the subject as a first dose (i.e., as an enteral dosage form; oral or rectal) and formulated for topical administration to the subject after the first dose (e.g., as a transdermal or transmucosal dosage form). In another embodiment, the peptides and compositions, as disclosed herein, are formulated for topical administration selected from the group consisting of transdermal and transmucosal administration. In a further embodiment, the peptides and compositions, as disclosed herein, are formulated for transdermal administration.

In yet another embodiment, it may be desirable to administer the peptides or compositions, as disclosed herein, enterally (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for administration as a first dose enterally and formulated for administration as a controlled release dosage form, wherein the controlled release dosage form is formulated for administration subsequent to the first dose. In an embodiment, the enteral dose is formulated for oral administration. In another embodiment, the controlled release dosage form is formulated for parenteral administration.

In an embodiment, it may be desirable to administer the peptides or compositions, as disclosed herein, topically (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for topical administration as a first dose and formulated for administration as a controlled release dosage form, wherein the controlled release dosage form is formulated for administration subsequent to the first topical dose. In an embodiment, the topical dose is formulated for transdermal administration. In another embodiment, the controlled release dosage form is formulated for parenteral administration.

The invention will now be described with reference to the following Examples which illustrate some preferred aspects of the present invention. However, it is to be understood that the particularity of the following description of the invention is not to supersede the generality of the preceding description of the invention.

### EXAMPLES

### Example 1: Case Study 1

The subject was a 50 year old female patient that suffers from an average of about one migraine per month. The migraine severity was described as incapacitating, and necessitating cessation of work. Usual rescue medication for previous migraine attacks included Panadol and imported codeine/caffeine product.

Dosage: 1 mg (four 0.25 mg capsules) LAT8881 (SEQ ID NO:2).

Main observations:
At the time of dosing, migraine pain was rated 8 out of 10 and nausea was rated 7 out of 10.
An hour after dosing, migraine pain was rated 7 out of 10 and nausea was rated 5 out of 10.
4 hours after dosing, migraine pain was rated 6 out of 10 and nausea was rated 3 out of 10.
No migraine was reported in the following month.

### Example 2: Case Study 2

The subject was a 25 year old female patient that suffers from frequent migraines, with an average of about two migraines per month. Migraine severity was described as incapacitating, and necessitating cessation of work and other daily activities. Usual rescue medication for previous migraine attacks included ibuprofen, Panadol and an imported codeine/caffeine product, which was reported by the subject to be losing their effectiveness.

Dosage: 1 mg (two 0.5 mg capsules) LAT8881 (SEQ ID NO:2)

Main observations:
At the time of dosing, migraine pain was rated 8 out of 10 and nausea was rated 8 out of 10.
An hour after dosing, migraine pain was rated 2 out of 10 and nausea was rated 2 out of 10.
4 hours after dosing, migraine pain was rated 1 out of 10 and nausea was rated 1 out of 10.
No migraines reported in the following two months.

### Example 3: Case Study 3

The subject was a female aged between 28-30 years old, that suffers from random (once every 3-6 months), but severe, migraines. The migraine severity is often described as incapacitating and necessitating cessation of work. Medical practitioners have described the subject as a unique patient in the terms of the severity and clustering of pain, resulting in very severe headache in certain areas.

Rescue medication for previous migraine attacks included strong over-the-counter analgesics and discontinued use of Topirimate (a beta blocker; which was effective with migraine pain, but had significant cognitive side effects for the subject). The subject was prescribed a regiment of regular botox injections, which was effective for a few months. The subject also reported that migraines have led to hospitalization for six-ten day stays, and on one occasion, required treatment with intravenous lignocaine for pain management. The subject also takes allegron (a tricyclic antidepressant).

Dosage: 0.5 mg (two 0.25 mg capsules) LAT8881 (SEQ ID NO:2)

Main observations:
At the time of dosing, migraine pain was rated 10 out of 10 and nausea was rated 0 out of 10.
Overnight, the migraine pain was rated 0 out of 10 and nausea was rated 0 out of 10.
No migraines reported in the following three months.

### Example 4: Case Study 4

The subject was a 45 year old female that suffers from migraines and occipital neuralgia. The subject presented with bilateral tenderness of both occipital nerve sites and bilateral headaches, bitemporally and behind the eyes, all indicative of a migraine attack.

Dosage: 0.4 mg of LAT8881 (SEQ ID NO:2) injected into the right greater occipital nerve.

Main observations:
The subject described a sensation of running water under the skin, with the migraine receding bilaterally from behind the eyes, and pain relief starting at the site of injection, running down the neck and then bilaterally.
Within ten minutes, the subject reported being pain-free, with no tenderness on either side over the occipital nerve. The subject also reported mental clarity and loss of brain fog.

The above examples show that LAT8881 (SEQ ID NO:2) is capable of treating migraine and alleviating the pain and nausea associated with migraine.

### Example 5: Case Study 5

The primary objective of this study was to evaluate the effect of oral LAT8881 on migraine headache compared with placebo, when assessed by the headache severity on an 11-point Numeric Rating Scale (NRS).

The secondary objectives of this study included:
a) to evaluate the effect of oral LAT8881 on migraine-associated symptoms compared with placebo, when assessed by the change in symptoms based on a 11-point Likert scale;
b) to investigate the percentage of subjects achieving “no headache pain” following treatment with oral LAT8881 compared with placebo at any time-point to 8 hours post dose, with no use of rescue medication; and
c) to evaluate the safety and tolerability of single doses of oral LAT8881 in subjects with migraine headache.

This was a randomised, placebo-controlled, double-blind, crossover, Phase Ha proof of concept study to investigate the efficacy and safety of oral LAT8881 (SEQ ID NO:2) in acute migraine headache with or without aura.

During the screening period, subjects kept a diary to record onset and duration of headaches, including pain and symptom scores and concomitant medications over a 4-5 week period. Female subjects of childbearing potential also record menstrual cycles. Subjects also nominated their most troublesome symptom.

Subjects entered into the study were randomised to receive IMP (LAT8881; 60 mg or placebo), taken at the onset (within one hour from the onset of pain) of a migraine headache of moderate to severe intensity [Numeric rating scale (NRS)≥4]. Subjects were advised not to take rescue medication until at least 2 hours post dose with LAT8881. Subjects were given one dose of IMP (2 capsules), to treat one migraine headache.

Subjects were asked to complete diaries to record menstrual cycle (in menstruating females), onset and nature of migraine and non-migraine headaches, pain scores at time of IMP dosing and various time points post dose, associated symptoms and rescue medication. Adverse events were monitored.

After treatment of one migraine headache (or a maximum 28 days), the subject was asked to return to the clinic for re-evaluation and crossover to the second treatment. In the second treatment period, subjects were asked to take IMP in the same manner as in treatment period 1.

A follow-up (End of study) visit occurred within 7 days after treatment of one migraine headache in the second treatment period or a maximum 8 weeks from randomisation.

Subjects were asked to complete diaries to record menstrual cycle (in menstruating females), onset and nature of migraine and non-migraine headaches, pain scores at time of IMP dosing and various time points post dose, associated symptoms and rescue medication. Adverse events were monitored.

### Sample Size Justfication

20 subjects were targeted for completion of the study, defined as taking one dose of IMP in each treatment period and attending the follow-up (End of study) visit.

Assuming up to 20% of subjects would not treat one migraine headache in each treatment period or would drop out of the study, 26 subjects were targeted for recruitment into the study.

In circumstances where the dropout rate or non-completion rate is higher than anticipated, additional subjects would be enrolled to ensure 20 subjects complete the study.

### Randomisation and Blinding

A computer-generated randomisation schedule and treatment allocation was prepared by an unblinded statistician prior to the start of the study. The treatment sequence each subject would receive would not be disclosed to the investigator, study site personnel, subjects, or sponsor personnel.

The randomisation codes would be available to the Investigator if required for emergency unblinding purposes.

### Inclusion/Exclusion Criteria

### A. Inclusion Criteria:

Subjects must have met the following criteria to be recruited into the study:

1. Males or females aged 18 to 75 years at the time of consent.
2. Diagnosis of episodic migraine headache at least 12 months ago with or without aura as defined in ICHD-3-beta.
3. Onset of migraine headache before age 50.
4. Medical history of 2-8 migraine headache attacks per month for the previous 12 months; ≥75% of attacks progress to moderate or severe pain within 2 hours (i.e., rapidly-escalating).
5. Minimum 48 hours on average between migraine headache attacks.
6. Acute headache medication on ≤14 days/month in the 3 months prior to screening.
7. Willing and able to comply with all study procedures including completion of a headache diary and a migraine diary on the day of a migraine headache.
8. Female subjects must be:

a) of non-child-bearing potential [surgically sterilised or postmenopausal (12 months with no menses without alternative medical cause)]; OR

b) not pregnant, breast feeding or planning to become pregnant AND willing to comply with the medically acceptable contraceptive requirements of the study from Screening to at least 28 days after the last IMP administration.

9. Male subjects with female partners of childbearing potential must use adequate and highly effective methods of contraception, from screening until 28 days after their last IMP administration.
10. Subjects must be sufficiently competent in English to understand the purposes and risks of the study and to provide written informed consent.

### B. Exclusion Criteria:

Subjects who met any of the following criteria would be excluded from the study:

1. Unable to distinguish migraine from other primary headache conditions.
2. Average of 15 or more headache (migraine or non migraine) days per month or history of more than 25% of headaches occurring at time of wakening (wake up headaches).
3. History of aura lasting more than 60 minutes.
4. History of vomiting within 2 hours of onset of a migraine headache in more than 25% of migraine headaches.
5. Medication overuse headache, defined as:

a. use of opioids, triptans or ergot alkaloids or any combination of these medications for treatment of headaches 10 or more days per month during the 90 days prior to screening; OR

b. Non-steroidal anti-inflammatory drugs (NSAIDs) or simple analgesics for treatment of headaches on more than 14 days per month during the 90 days prior to screening.

6. Recent (3 years) history of frequent or chronic hemiplegic/basilar migraine, tension headache, retinal migraine, ophthalmoplegic migraine as per ICHD classification, or treatment resistant atypical migraine.
7. Hospital admission for status migrainosis or medication overuse headache within 6 months of screening.
8. Current clinically significant systemic disease or neurological or psychiatric condition which in the opinion of the investigator or sponsor could jeopardise the safety of the subject or the validity of the study results.
9. Cerebrovascular disease, including but not limited to a history of stroke or recent (3 years) transient ischaemic attack (TIA).
10. Major surgery within 6 weeks of screening or planned during the study period
11. Clinically significant abnormality as assessed by the investigator or sponsor's medical monitor on haematology, biochemistry, vital signs, physical examination or 12-lead electrocardiogram (ECG).
12. Malignancy within 5 years of screening, with the exception of carcinoma in situ, non-melanoma skin cancers and prostate cancer not requiring treatment or on stable (>6 months) treatment with hormone therapy.
13. History of alcohol abuse, illicit or illegal drug use in the last 2 years
14. Use of Prohibited medications or treatments within the specified time period before Screening or planned during the study.
15. Participation in another clinical trial or administration of any investigational product or experimental product within 60 days or 5 half-lives (whichever is longer).
16. History of significant hypersensitivity to LAT8881, excipients in the drug product formulation or drugs of a similar chemical or pharmacological class.
17. Surgical or medical conditions which could significantly alter drug absorption, distribution, metabolism or excretion.
18. An employee of the sponsor or research site personnel directly affiliated with this study, whether biological or legally adopted, or their immediate family members, defined as a spouse, parent, sibling, or child.

### Treatment Allocation

Subjects entered into the study were randomised to receive IMP (LAT8881; 60 mg or placebo; orally), to be taken at the onset (within one hour from the onset of pain) of a migraine headache of moderate to severe intensity.

After treatment of one migraine headache (or a maximum 28 days), the subject was asked to return to the clinic for re-evaluation and crossover to the second treatment.

### Primary Efficacy Endpoints

Primary efficacy endpoints included a change in migraine headache pain score, using an 11-point NRS, (0=none, 10=worst imaginable), from the time of dosing to 0.5, 1.0, 1.5, 2, 4, 8 and 24 hours post dose. Pain was recorded in the subject' diary and was expected to reflect the subject's pain at the time of recording.

### Secondary Efficacy Endpoints

Secondary efficacy endpoints included:
a) Change in migraine-associated symptoms of nausea, photophobia and phonophobia. Symptoms were assessed on an 11-point Likert scale with 0=no symptoms and 10 being severe symptoms at time of dosing and at 0.5, 1.0, 1.5, 2, 4, 8 and 24 hours post dose;
b) Change in each subject's most troublesome symptom that may include nausea, photophobia and phonophobia cognitive impairment, dizziness and functional disability. Symptoms were assessed on an 11-point Likert scale with 0=no symptoms and 10 being severe symptoms at time of dosing and at 0.5, 1.0, 1.5, 2, 4, 8 and 24 hours post dose; and
c) The percentage of subjects achieving “no headache pain” at 0.5, 1.0, 1.5, 2, 4 and 8 hours post dose.

The responder rates are shown in Table 2.

### Results

Twenty-one subjects were enrolled in the study. Of those, 3 subjects withdrew from the study before completion, one subject was non-compliant at one treatment, one subject received only one treatment with LAT8881 and another subject received only one treatment with placebo.

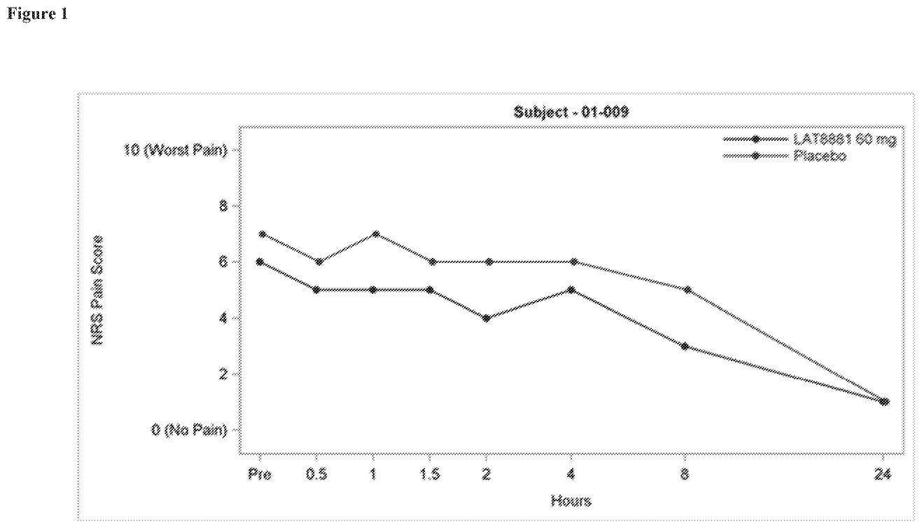

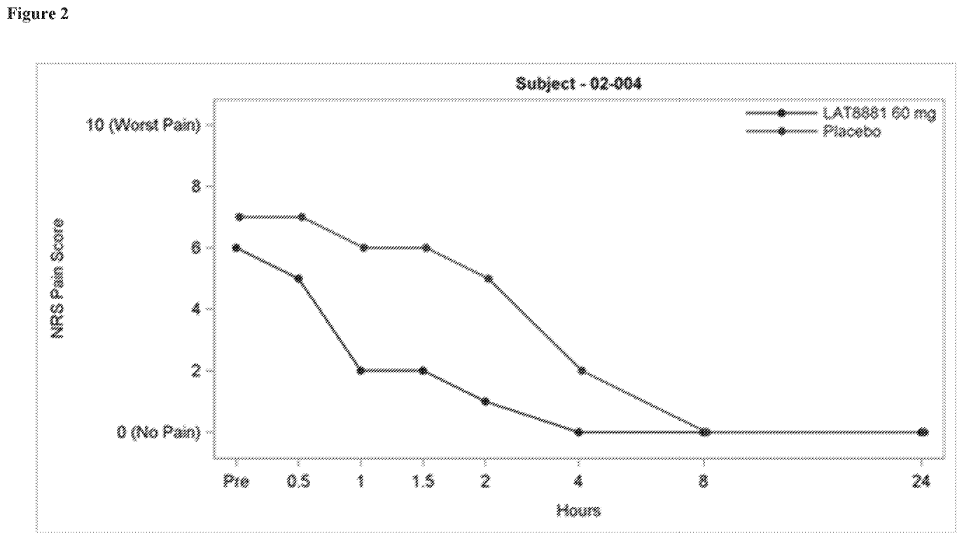

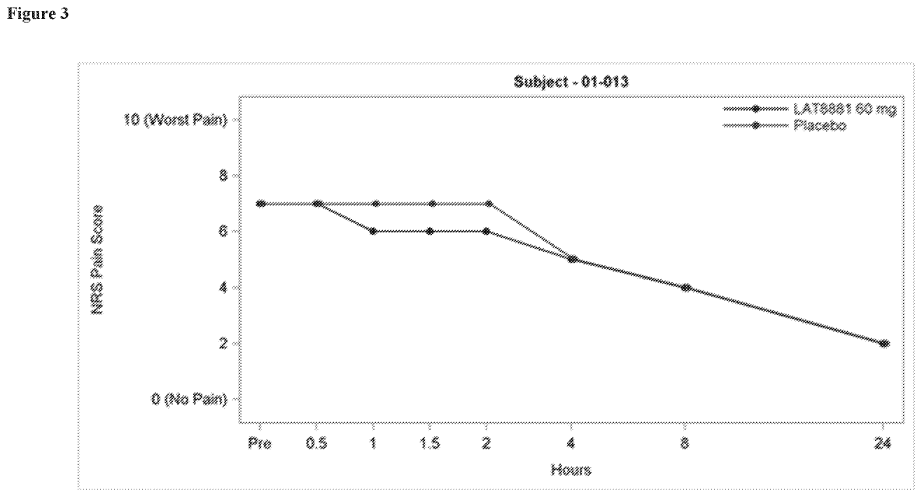

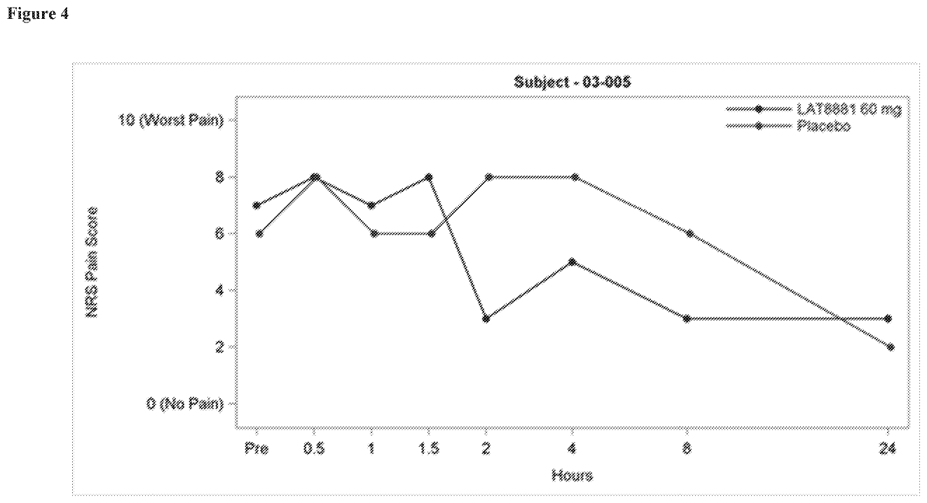

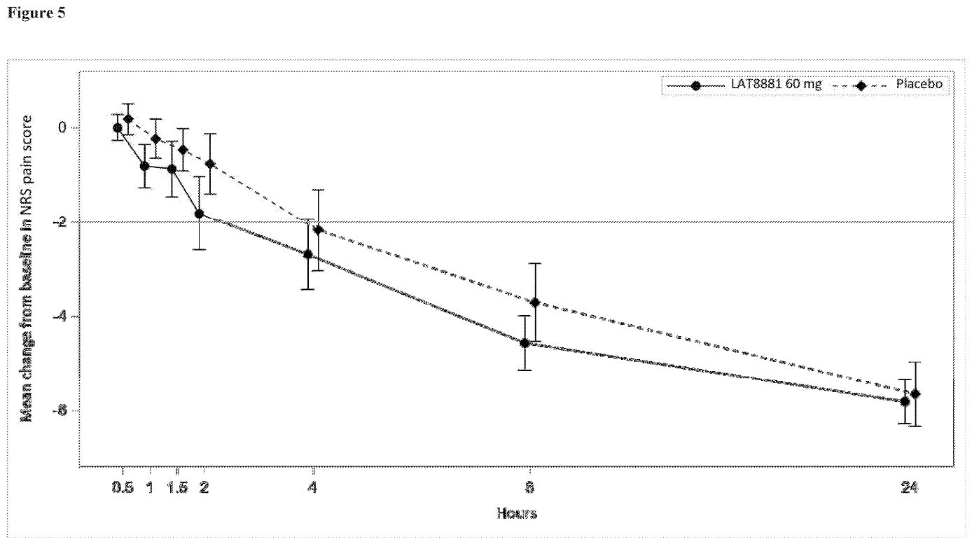

Post-hoc analyses were undertaken to assess the responder rate at each time point after dose of IMP. Two classifications of responder were defined: at least 30% change from baseline in NRS score and an NRS score of less than or equal to 3 at the post dose time points. After unblinding, it was discovered that one subject had recorded a pre-dose NRS score of 2 in period 1, a major protocol deviation as the instructions were to take IMP within one hour of the onset of a migraine headache of moderate to severe intensity (NRS>=4). This subject's data for this period was excluded. The responder rate for each treatment and difference between LAT8881 and placebo (95% confidence interval) at each time point are provided in Table 2, below (P1=Period 1; P2=Period 2; Photo=Photophobia; Phono=Phonophobia). FIGS. 1-4 show selected examples of NSR Pain Scores of subjects that completed the study and who received treatment with either placebo or with LAT8881 (SEQ ID NO:2; 60 mg per dose, orally).

TABLE 2
Responder rates assessed by at least 30% change from baseline and an NRS score of ≤ 3 at each post-dose time point
for LAT8881 60 mg, placebo and the difference between LAT8881 and placebo with 95% confidence limits

| | | | 30% change from baseline | | NRS Score <= 3 | |
|---|---|---|---|---|---|---|
| Timepoint | | Treatment | Count/Total | Responder rate (95% CI) | Count/Total | Responder rate (95% CI) |
| 30 | minutes | LAT8881 60 mg | 0/16 | 0 | 0/16 | 0 |
| | | Placebo | 0/16 | 0 | 0/16 | 0 |
| | | LAT8881 60 mg-Placebo | | Not estimable | | Not estimable |
| 1 | hour | LA18881 60 mg | 2/16 | 12.50 | 1/16 | 6.25 |
| | | Placebo | 0/16 | 0 | 1/16 | 6.25 |
| | | LAT8881 60 mg-Placebo | — | 12.5 (−3.7, 28.7) | | 0 (−16.77, 16.77) |
| 1.5 | hours | LA18881 60 mg | 3/16 | 18.75 | 3/16 | 18.75 |
| | | Placebo | 1/16 | 6.25 | 1/16 | 6.25 |
| | | LA18881 60 mg-Placebo | | 12.5 (−10, 35) | | 12.5 (−10, 35) |
| 2 | hours | LAT8881 60 mg | 8/16 | 50.00 | 5/16 | 31.25 |
| | | Placebo | 5/16 | 31.25 | 1/16 | 6.25 |
| | | LAT8881 60 mg-Placebo | | 18.75 (−14.66, 52.16) | | 25 (-0.62, 50.62) |
| 4 | hours | LAT8881 60 mg | 8/16 | 50.00 | 5/16 | 31.25 |
| | | Placebo | 8/16 | 50.00 | 6/16 | 37.50 |
| | | LAT8881 60 mg-Placebo | | 0 (−34.65, 34.65) | | −6.25 (-39.09, 26.59) |
| 8 | hours | LAT8881 60 mg | 14/16 | 87.50 | 13/16 | 81.25 |
| | | Placebo | 10/16 | 62.50 | 9/16 | 56.25 |
| | | LAT8881 60 mg-Placebo | | 25 (−3.73, 53.73) | | 25 (−5.93, 55.93) |
| 24 | hours | LAT8881 60 mg | 16/16 | 100.00 | 15/16 | 93.75 |
| | | Placebo | 14/16 | 87.50 | 14/16 | 87.50 |
| | | LAT8881 60 mg-Placebo | | 12.5 (−3.7, 28.7) | | 6.25 (−13.83, 26.33) |

Analysis excludes subject 03-014, period 1 who had a major protocol deviation with a pre-dose NRS score of 2 (protocol requirement ≥ 4)

Of the 15 subjects who completed the study, the following observation were made:
a) For the subjects who received treatment with LAT8881, 9 showed a benefit at 2 hours [median 3, range 1-10], 5 were slightly worse [median increase 2, range 1-2] and 1 was unchanged. Conversely, for subjects who received the placebo, 8 showed a benefit at 2 hours [median 2, range 1-4], 4 were worse [median 4.5, range 1-6] and 3 were unchanged;
b) Of the subjects whose pain had reduced to 3 or less after 2 hours of receiving treatment, 4 subjects received LAT8881 and only one received placebo;
c) Of the subjects who were menstruating at the time of the migraine, 2 out of 2 who received LAT8881 benefited with an average score reduction of 6, range 10 to 2, whereas of the 3 subjects who received placebo when menstruating, only 1 benefited, one was unchanged and the third was worse. These important benefits are supported by the fact that one of the subjects was menstruating at the time of each treatment. The LAT8881 resulted in a benefit of a reduction of 2 whereas the placebo led to an increase of 3.
   These observations are set out in Table 3.

TABLE 3

| | | | | | | | | | | | Pla- | |
|---|---|---|---|---|---|---|---|---|---|---|---|---|
| | | | | | | | | | | | cebo | |
| | | | | | | | Pla- | Pla- | | | Mi- | |
| | | Per- | | | | Pla- | cebo | cebo | | LAT8881 | graine | |
| | | proto- | | LAT8881 | LAT8881 | cebo | pain | change | Time | Migraine | during | Nausea |
| | LAT8881 | col | LAT8881 | pain | change | pre- | score | from | since | during | men- | change |
| Subject | Period 1 | (P1- | predose | score at | from | dose | at 2 | base- | diag- | menstru- | stru- | 2 hrs |
| ID | or 2 | P2) | pain | 2 hours | baseline | pain | hours | line | nosis | ation | ation | LAT8881 |
| 01-007 | 1 | N-N | | | | | | | 12 | | | |
| 01-008 | 1 | Y-N | 5 | 2 | −3 | | | | 24 | | | 0 |
| 02-004 | 1 | Y-Y | 6 | 1 | −5 | 7 | 5 | −2 | 5 | | | −5 |
| 02-009 | 1 | N-N | | | | | | | 19 | | | |
| 02-011 | 1 | N-N | | | | | | | 26 | | | |
| 02-014 | 1 | Y-Y | 10 | 0 | −10 | 10 | 6 | −4 | 16 | yes | | −10 |
| 02-016 | 1 | Y-Y | 4 | 5 | 1 | 4 | 2 | −2 | 4 | | | 1 |
| 03-002 | 1 | Y-Y | 6 | 8 | 2 | 8 | 8 | 0 | 1 | | | −1 |
| 03-004 | 1 | N-Y | 9 | 6 | −3 | 9 | 5 | −4 | 6 | | | −2 |
| 03-005 | 1 | Y-Y | 7 | 3 | −4 | 6 | 8 | 2 | 22 | | | −1 |
| 01-009 | 2 | Y-Y | 6 | 4 | −2 | 7 | 6 | −1 | 29 | | | −1 |
| 01-013 | 2 | Y-Y | 7 | 6 | −1 | 7 | 7 | 0 | 43 | | | −1 |
| 02-003 | 2 | Y-Y | 7 | 5 | −2 | 7 | 5 | −2 | 11 | | | −2 |
| 02-006 | 2 | Y-Y | 6 | 7 | 1 | 5 | 5 | 0 | 26 | | yes | 2 |
| 02-010 | 2 | Y-Y | 5 | 3 | −2 | 4 | 7 | 3 | 19 | yes | yes | −8 |
| 02-012 | 2 | Y-N | | | | 8 | 5 | −3 | 36 | | yes | |
| 02-015 | 2 | Y-Y | 8 | 9 | 1 | 8 | 9 | 1 | 25 | | | 3 |
| 02-018 | 2 | Y-Y | 9 | 6 | −3 | 7 | 4 | −3 | 5 | | | −3 |
| 03-011 | 2 | Y-Y | 10 | 6 | −4 | 8 | 6 | −2 | 4 | | | −5 |
| 03-012 | 2 | Y-Y | 8 | 8 | 0 | 7 | 5 | −2 | 10 | | yes | 0 |
| 03-014 | 2 | Y-Y | 6 | 8 | 2 | 2 | 8 | 6 | 41 | | | 6 |

| | Nau- | | | | | Most | Most | | | | |
|---|---|---|---|---|---|---|---|---|---|---|---|
| | sea | | | | Phono | Trouble- | Trouble- | | | | |
| | change | Photo | Photo | Phono | change | some, | some, | | | | |
| | 2 hrs | change | change | change | at 2 | change | change | | Aura? | | Headaches |
| Subject | Pla- | at 2 hrs | at 2 hrs | at 2 hrs | hrs | 2 hrs | 2 hrs | Aura? | Pla- | | per |
| ID | cebo | LAT8881 | Placebo | LAT8881 | Placebo | LAT8881 | Placebo | LAT8881 | cebo | Age | month |
| 01-007 | | | | | | | | | | 30 | 5 |
| 01-008 | | 0 | | 0 | | −5 | | no | | 60 | 5 |
| 02-004 | 1 | −2 | 0 | 0 | 0 | −5 | 1 | no | no | 34 | 6 |
| 02-009 | | | | | | | | | | 38 | 2 |
| 02-011 | | | | | | | | | | 43 | 3 |
| 02-014 | −5 | −7 | −6 | −7 | −8 | −10 | −4 | ves | yes | 36 | 8 |
| 02-016 | 0 | | −2 | 4 | −2 | 3 | −2 | yes | yes | 31 | 4 |
| 03-002 | 0 | 1 | 1 | 2 | 1 | 0 | 0 | yes | yes | 50 | 4 |
| 03-004 | −3 | −6 | −4 | 0 | −6 | −4 | −4 | no | no | 32 | 8 |
| 03-005 | −2 | −6 | 5 | −4 | −4 | 1 | −2 | yes | yes | 34 | 7 |
| 01-009 | 0 | −2 | 3 | −1 | 1 | −5 | 1 | no | no | 67 | 4 |
| 01-013 | −1 | −2 | −1 | 0 | 0 | −2 | −1 | yes | yes | 55 | 7 |
| 02-003 | 0 | −2 | −1 | −2 | 0 | −1 | −2 | no | no | 32 | 3 |
| 02-006 | −1 | 1 | −1 | 0 | 0 | 0 | −1 | no | no | 45 | 6 |
| 02-010 | 2 | 0 | 2 | 0 | 1 | 0 | 2 | no | no | 33 | 4 |
| 02-012 | −4 | | 0 | | −1 | | −3 | no | no | 46 | 2 |
| 02-015 | 1 | 1 | 0 | 2 | 0 | 3 | 1 | no | no | 38 | 4 |
| 02-018 | −4 | −2 | −2 | 0 | 0 | −2 | −3 | no | no | 33 | 4 |
| 03-011 | −1 | −4 | −2 | 0 | −5 | −2 | −3 | yes | yes | 20 | 4 |
| 03-012 | −1 | 0 | −3 | 0 | −2 | 0 | −9 | yes | yes | 30 | 4 |
| 03-014 | 1 | 1 | 6 | 3 | 4 | 3 | 6 | no | no | 54 | 5 |

The claims defining the invention are as follows:

1. A method of treating migraine in a subject, the method comprising administering to a subject a therapeutically effective amount of a peptide of formula (I), or a pharmaceutically acceptable salt thereof:
(SEQ ID NO: 1)
R¹-CRSVEGSCG-R² (I)
wherein
R¹ is selected from the group consisting of YLRIVQ, LRIVQ, RIVQ, IVQ, VQ, and Q, or R¹ is absent; and
R² is F (phenylalanine) or R² is absent.

2. The method of claim 1, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

3. The method of claim 2, wherein the peptide is YLRIVQCRSVEGSCGF (SEQ ID NO:2).

4. The method of claim 2, wherein the peptide is CRSVEGSCG (SEQ ID NO:4).

5. The method of claim 2, wherein the peptide is CRSVEGSCGF (SEQ ID NO:5).

6. The method of any one of claims 1 to 5, wherein the subject is a human.

7. A pharmaceutical composition comprising a peptide of formula (I), or a pharmaceutically acceptable salt thereof, for use in the treatment of migraine in a subject:
(SEQ ID NO: 1)
R¹-CRSVEGSCG-R² (I)
wherein
R¹ is selected from the group consisting of YLRIVQ, LRIVQ, RIVQ, IVQ, VQ, and Q,
or R¹ is absent; and
R² is F (phenylalanine), or R² is absent.

8. The composition for use according to claim 7, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

9. The composition for use according to claim 8, wherein the peptide is YLRIVQCRSVEGSCGF (SEQ ID NO:2).

10. The composition for use according to claim 8, wherein the peptide is CRSVEGSCG (SEQ ID NO:4).

11. The composition for use according to claim 8, wherein the peptide is CRSVEGSCGF (SEQ ID NO:5).

12. The composition for use according to any one of claims 7 to 11, wherein the subject is a human.

13. Use of a peptide of formula (I), or a pharmaceutically acceptable salt thereof, in the manufacture of a medicament for the treatment of migraine in a subject:
(SEQ ID NO: 1)
R¹-CRSVEGSCG-R² (I)
wherein
R¹ is selected from the group consisting of YLRIVQ, LRIVQ, RIVQ, IVQ, VQ, and Q, or R¹ is absent; and
R² is F (phenylalanine), or R² is absent.

14. Use of claim 13, wherein the peptide is selected from the group consisting of
(SEQ ID NO: 2)
YLRIVQCRSVEGSCGF,
(SEQ ID NO: 3)
LRIVQCRSVEGSCGF,
(SEQ ID NO: 4)
CRSVEGSCG
and
(SEQ ID NO: 5)
CRSVEGSCGF.

15. Use of claim 14, wherein the peptide is YLRIVQCRSVEGSCGF (SEQ ID NO:2).

16. Use of claim 14, wherein the peptide is CRSVEGSCG (SEQ ID NO:4).

17. Use of claim 14, wherein the peptide is CRSVEGSCGF (SEQ ID NO:5).

18. Use of any one of claims 13 to 17, wherein the subject is a human.

19. A method of treating migraine in a subject, the method comprising administering to a subject a therapeutically effective amount of a peptide of formula (II), or a pharmaceutically acceptable salt thereof:
(SEQ ID NO: 6)
R¹-CRRFVESSC-R² (II)
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

20. The method of claim 19, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

21. The method of claim 20, wherein the peptide is YLRVMKCRRFVESSCAF (SEQ ID NO:7).

22. The method of claim 20, wherein the peptide is CRRFVESSCAF (SEQ ID NO:9).

23. The method of claim 20, wherein the peptide is CRRFVESSCA (SEQ ID NO:10).

24. The method of any one of claims 19 to 23, wherein the subject is selected from the group consisting of a feline, a canine and an equine.

25. A pharmaceutical composition comprising a peptide of formula (II), or a pharmaceutically acceptable salt thereof, for use in the treatment of migraine in a subject:
(SEQ ID NO: 6)
R¹-CRRFVESSC-R² (II)
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

26. The composition for use according to claim 25, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

27. The composition for use according to claim 26, wherein the peptide is YLRVMKCRRFVESSCAF (SEQ ID NO:7).

28. The composition for use according to claim 26, wherein the peptide is CRRFVESSCAF (SEQ ID NO:9).

29. The composition for use according to claim 26, wherein the peptide is CRRFVESSCA (SEQ ID NO:10).

30. The composition for use according to any one of claims 19 to 29, wherein the subject is selected from the group consisting of a feline, a canine and an equine.

31. Use of a peptide of formula (II), or a pharmaceutically acceptable salt thereof, in the manufacture of a medicament for the treatment of migraine in a subject:
(SEQ ID NO: 6)
R¹-CRRFVESSC-R² (II)
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

32. Use of claim 31, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

33. Use of claim 32, wherein the peptide is YLRVMKCRRFVESSCAF (SEQ ID NO:7).

34. Use of claim 32, wherein the peptide is CRRFVESSCAF (SEQ ID NO:9).

35. Use of claim 32, wherein the peptide is CRRFVESSCA (SEQ ID NO:10).

36. Use of any one of claims 31 to 35, wherein the subject is selected from the group consisting of a feline, a canine and an equine.

37. A method of treating migraine in a subject, the method comprising administering to a subject a therapeutically effective amount of a peptide of formula (III):
(III)
(SEQ ID NO: 11)
R¹-C-R-X¹-X²-P-X³-X⁴-X⁵-X⁶-C-R²
wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,
or R¹ is absent; and
R² is selected from the group consisting of
S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,
or R² is absent;
or a pharmaceutically acceptable salt thereof.

38. The method of claim 37, wherein the peptide of formula (III) has an amino acid sequence selected from the group consisting of:
(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

39. A pharmaceutical composition comprising a peptide of formula (III), or a pharmaceutically acceptable salt thereof, for use in the treatment of migraine in a subject:
R¹-C-R-X¹-X²-P-X³-X⁴-X⁵-X-C-R² (III) (SEQ ID NO:11)
wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,
or R¹ is absent; and
R is selected from the group consisting of
S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,
or R² is absent.

40. The composition of claim 39, wherein the peptide of formula (III) has an amino acid sequence selected from the group consisting of:
(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

41. Use of a peptide of formula (III), or a pharmaceutically acceptable salt thereof, in the manufacture of a medicament for the treatment of migraine in a subject:
(III)
(SEQ ID NO: 11)
R¹-C-R-X¹-X²-P-X³-X⁴-X⁵-X⁶-C-R²
wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,
or R¹ is absent; and
R² is selected from the group consisting of
S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,
or R² is absent.

42. Use of claim 41, wherein the peptide of formula (III) has an amino acid sequence selected from the group consisting of:
(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

43. A method of treating migraine in a subject, the method comprising administering to a subject in need thereof a peptide of formula (IV) or a pharmaceutically acceptable salt thereof:
(IV)
(SEQ ID NO: 40)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL,
K or R¹ is absent; and
R² is G (glycine), or R² is absent.

44. The method of claim 43, wherein the peptide of formula (IV) is selected from the group consisting of CRIIHNNNC (SEQ ID NO:41), CRIIHNNNCG (SEQ ID NO:42), CRIVYDSNC (SEQ ID NO:43) and CRIVYDSNCG (SEQ ID NO:44).

45. A pharmaceutical composition comprising a peptide of formula (IV) or a pharmaceutically acceptable salt thereof, for use in the treatment of migraine in a subject:
(IV)
(SEQ ID NO: 40)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL,
K or R¹ is absent; and
R² is G (glycine), or R² is absent.

46. The composition of claim 45, wherein the peptide of formula (IV) is selected from the group consisting of CRIIHNNNC (SEQ ID NO:41), CRIIHNNNCG (SEQ ID NO:42), CRIVYDSNC (SEQ ID NO:43) and CRIVYDSNCG (SEQ ID NO:44).

47. Use of a peptide of formula (IV) or a pharmaceutically acceptable salt thereof, in the manufacture of a medicament for the treatment of migraine in a subject:
(IV)
(SEQ ID NO: 40)
R¹-C-R-I-X₁-X₂-X₃-X₄-N-C-R²
wherein
X₁ is an amino acid residue selected from isoleucine (1) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL,
K or R¹ is absent; and
R² is G (glycine), or R² is absent.

48. The use of claim 47, wherein the peptide of formula (IV) is selected from the group consisting of CRIIHNNNC (SEQ ID NO:41), CRIIHNNNCG (SEQ ID NO:42), CRIVYDSNC (SEQ ID NO:43) and CRIVYDSNCG (SEQ ID NO:44).
